(19) United States
(12) Patent Application Publication
(10) Pub. No.: US 2024/0182955 A1
(43) Pub. Date: Jun. 6, 2024

(54) DNA FRAGMENT JOINING DETECTING METHOD AND KIT THEREOF

(71) Applicant: ACT GENOMICS (IP) LIMITED, Hong Kong City (HK)

(72) Inventors: AN HAU, Taipei (TW); PEI-YI LIN, Taipei (TW); DATSEN GEORGE WEI, Taipei (TW); HUA-CHIEN CHEN, Taipei (TW); SHU-JEN CHEN, Taipei (TW); CHIH-YI YANG, Taipei (TW)

(21) Appl. No.: 18/546,431

(22) PCT Filed: Feb. 17, 2022
(86) PCT No.: PCT/US2022/016877

§ 371 Date: Aug. 15, 2023

Related U.S. Application Data
(60) Provisional application No. 63/150,095, filed on Feb. 17, 2021.

(51) Int. Cl. C12Q 1/6827; C12Q 1/686

(52) U.S. Cl. CPC C12Q 1/6827; C12Q 1/686; C12Q 2600/156

(57) ABSTRACT

The present disclosure relates to the fields of a method a kit for molecular diagnostics and genomics. More particularly, this disclosure relates to a method and a kit fir detecting a DNA fragment joining event or distinguishing an alternative splicing event. The present disclosure also relates to a method for administering a subject with proper treatment by steps of determining the risk of a particular cancer type or genotype.

### CROSS-REFERENCE TO RELATED APPLICATIONS

This application claims priority of Provisional Application No. 63/150,095, filed on Feb. 17, 2021, and the content of which are incorporated herein in its entirety by reference.

### REFERENCE TO A “SEQUENCE LISTING”, A TABLE, OR A COMPUTER PROGRAM LISTING APPENDIX SUBMITTED ON A COMPACT DISC AND AN INCORPORATION-BY-REFERENCE OF THE MATERIAL ON THE COMPACT DISC

The instant application contains a Sequence Listing which has been submitted electronically in ASCII format and is hereby incorporated by reference in its entirety. Said ASCII copy, created Jan. 13, 2022, is named “ACTG-9PCT_ST25.txt” and is 16,384 bytes in size.

### BACKGROUND OF THE INVENTION

The present invention relates to the fields of a method and a kit for molecular diagnostics and genomics. More particularly, this invention relates to a method and a kit for detecting a DNA fragment joining event or distinguishing an alternative splicing event. The present invention also relates to a method for administering a subject with proper treatment by steps of determining the risk of a particular cancer type or genotype.

In genetics, rearrangement, translocation, tandem repeat, inversion, insertion, deletion, or other chimeric variation of DNA, which is present on DNA fragment joining, is often synonymous with disease. These kinds of DNA damage in our cells of the organ have been proved lead to the starting point of a genetic disease or cancer; therefore, detecting DNA fragment joining can be used in screening for having potential health disorders or diseases. However, it is still not far away enough for precision medicine.

Constitutive splicing is introns removed and exons joined together in the process of RNA splicing. Alternative splicing is a deviation from normal splicing where the exons are skipped, introns are retained, the exons are mutually exclusive, or the alternative 5′ splice sites or alternative 3′ splice sites are conserved in mature mRNAs. Recently alternative splicing has become of interest because of its roles in gene expression and association with disease. For example, many introns retention events can be detected in the cytoplasm of primary cancer cells and be associated with the diversity of cancer cell transcriptomes.

DNA fragment joining and alternative splicing events have impact effects on the protein produced and may significantly affect disease risk, disease progression, and drug responses. Detecting DNA fragment joining or distinguishing these genetic variations alternative splicing can be used as diagnostic markers and it may be important for future target treatment in gene-related diseases. The correlation between alternative gene splicing and cancer drug resistance has been shown as discussed in Wang, Bi-Dar, and Norman H. Lee. “Aberrant RNA splicing in cancer and drug resistance.” Cancers 10.11 (2018): 458., incorporated herein by reference.

The identification of DNA fragment joining events or specific alternative splicing events can be through bioinformatic analysis, including next-generation sequencing (NGS), immunohistochemistry (IHC), fluorescent in situ hybridization (FISH), and either qRT-PCR, microarrays, or RNAseq data analysis. While NGS provides comprehensive information with details, it is not only costly and time-consuming but also requires more samples, thus limiting its clinical application. IHC detects the presence of produced proteins, but it is challenging to distinguish the relationship between genotype mutations and phenotype variations. FISH can detect gene fusions, but it requires separate reactions for detecting each fusion type and also requires highly-trained specialists to analyze the results. Thus, new, more accurate, and comprehensive methods are needed to detect DNA fragment joining types and predict the genotype, in particular the mRNA splicing defect which has been revealed associated with a characteristic feature of the disease.

### SUMMARY OF THE INVENTION

The present disclosure provides a method for detecting a DNA fragment joining event, and the method includes steps of:
(a) obtaining a DNA or a DNA from an extracted RNA in a sample;
(b) enriching the DNA with a set of oligonucleotides to obtain a target nucleic acid;
(c) probing the target nucleic acid with a split probe including:
(i) a first split probe being complementary to the 3′ end of a partner DNA fragment, a second split probe being complementary to the 5′ end of a target DNA fragment, and/or a third split probe being complementary to a third DNA fragment, wherein a gap of the first and second split probes targeting sites on the target nucleic acid is within 0-80 bp; or
(ii) a first split probe being complementary to the 5′ end of a partner DNA fragment, a second split probe being complementary to the 3′ end of a target DNA fragment, and/or a third split probe being complementary to a third DNA fragment, wherein a gap of the first and second split probes targeting sites on the target nucleic acid is within 0-80 bp; and
(d) detecting a signal that reflects a binding between the split probe and the target nucleic acid.

According to the above, the set of oligonucleotides is a gene-specific primer or a gene-specific probe.

According to the above, the DNA is amplified by multiplex PCR with at least two pairs of a gene-specific primers in step (b).

According to the above, the method further includes a step for determining
(i) the partner DNA fragment as an upstream DNA fragment and/or the target DNA fragment as a downstream DNA fragment through confirming the signal based on the first split probe binding to the 3′ end of the partner DNA fragment and/or the second split probe binding to the 5′ end of the target DNA fragment;
(ii) the partner DNA fragment as a downstream DNA fragment and/or the target DNA fragment as an upstream DNA fragment through confirming the signal based on the first split probe binding to the 5′ end of the partner DNA fragment and/or the second split probe binding to the 3′ end of the target DNA fragment or
(iii) whether or not the third DNA fragment is joined with the partner DNA fragment and the target DNA fragment through confirming the signal based on the third split probe binding to the third DNA fragment and a result of the target nucleic acid from an independent PCR.

According to the above, at least two pairs of the gene-specific primers are designed to obtain the target nucleic acid from the partner DNA fragment as an upstream DNA fragment.

According to the above, at least two pairs of the gene-specific primers are designed to obtain the target nucleic acid from the partner DNA fragment as a downstream DNA fragment.

According to the above, the gene-specific primer targets a DNA fragment joining boundary.

According to the above, the gene-specific primer targets within a distance of 0-80 bp from a DNA fragment joining boundary.

According to the above, the first split probe and the second split probe target within a distance of 0-40 bp from a DNA fragment joining boundary.

According to the above, the first split probe is selected from the group consisting of SEQ ID Nos: 32, 35, and any complementary sequence thereof.

According to the above, the second split probe is selected from the group consisting of SEQ ID Nos: 33, 36, and any complementary sequence thereof.

According to the above, the third split probe is selected from the group consisting of SEQ ID Nos: 32, 33, 35, 36, and any complementary sequence thereof.

According to the above, a length of the split probe is 10-60 bp.

According to the above, the target nucleic acid is probed with a split probe and a single probe targeting a DNA fragment joining boundary in step (c) of the method.

According to the above, the partner DNA fragment includes a sequence of a partner gene selected from the group consisting of ACVR2A, AFAP1, AFF1, AGAP3, AGBL4, AGGF1, AKAP13, AKAP6, AKAP9, AMOTL2, ANKRD11, APIP, ARGLU1, ARHGEF11, ARHGEF2, ATG7, ATP1B, BAG4, BAIAP2L1, BCAN, BCL6, BCR, BICC1, BRD3, BRD4, BTBD1, CAPZA2, CBR4, CCDC170, CCDC6, CD74, CDK12, CDK5RAP2, CEL, CEP170, CFB, CHTOP, CLCN6, CLIP1, CLIP2, CLTC, CNIH4, CNTRL, COL25A1, COX5A, CPD, CREBBP, CTRC, CTTN, CUX1, CYSTM1, DAB2IP, DAZL, DCTN1, DLG1, DNAJC7, DNAJC8, EIF3E, ELL, EML1, EML4, ENO1, EPHB2, EPS15, ERC1, ESRP1, ETV6, EZR, FAM131B, FAT1, FCGRT, FGFR1, FGFR3, FIP1L1, FKBP10, FN1, FNDC3B, FRY, FUS, GKAP1, GOLGA4, GON4L, GOPC, GRB7, GRHL2, GRIPAP, GSE1, GTF2E2, GTF2IRD1, HACL1, HIP1, HNRNPA2B1, IKZF2, IKZF3, IQSEC1, IRF2BP2, JAK2, KANK1, KCTD16, KCTD8, KHDRBS1, KIAA1549, KIF5B, KRT20, KRT39, KRTAP1-4, KTN1, LIPI, LMNA, LMNTD1, LRRC71, LRRFIP1, LTBP4, LYN, MAD2L2, MAGI3, MBIP, MBNL1, MED1, MEF2D, MET, MIR548F1, MKRN1, MLLT1, MLLT10, MLLT11, MLLT3, MLLT4, MPRIP, MRPL24, MSN, MTSS1, MUC2, MYH9, MYO5A, NACC2, NAV1, NBPF20, NCOA4, NFASC, NOS1AP, NRG1, NRIP1, NTRK1, NTRK2, NTRK3, P2RX5, P2RY8, PAIP1, PAN3, PAPD7, PARN, PDE4DIP, PDGFRA, PDGFRB, PEAR1, PGAP3, PHC3, PHF20, PICALM, PLEKHA6, PML, POLD4, PPFIBP1, PPL, PPP1R1B, PRDM16, PRDX1, PRDX4, PRKAR1A, PRKAR1B, PRKAR2A, PRPSAP1, PSMB3, PTPRR, PTPRZ1, QKI, RAC1, RALGPS2, RANBP2, RBPMS, RET, RFWD2, RNF213, ROS1, RRBP1, SATB1, SCAF11, SCP2, SCYL3, SDC4, SEC31A, SEP6, SEP9, SHC1, SHKBP1, SIL1, SLC34A2, SLC39A11, SLC45A3, SLC4A4, SLMAP, SMIM18, SND1, SPECC1L, SPTBN1, SPTBN2, SQSTM1, SRCIN1, SRGAP3, SSBP2, STK11IP, STRN, STRN3, TACC3, TADA2A, TATDN1, TBC1D2, TBL1XR1, TFG, TIMP3, TKT, TLE4, TMEM106B, TMEM40, TMPRSS2, TNS3, TP53, TPM3, TPM4, TPR, TRAF2, TRAK1, TRIM24, TRIM33, TRIM4, TRIM63, UBE2D2, UBE2R2, UFD1, USP13, VANGL2, VCAN, VCL, VIM, VPS18, WHSC1L1, WIPF2, WNK2, XBP1, ZAN, ZBTB7B, ZNF710, and ZPR1.

According to the above, the target DNA fragment includes a sequence of a target gene selected from the group consisting of ABL, AKT3, ALK, AXL, BCR, BRAF, CD74, ERBB2, ERBB4, ERG, ESR1, ETV1, ETV4, ETV5, ETV6, EZR, FGFR1, FGFR2, FGFR3, KIT, KMT2A, MET, NRG1, NRG2, NTRK1, NTRK2, NTRK3, NUTM1, PDGFRA, PDGFRB, PIK3CA, RAF1, RARA, RET, ROS1, RSPO2, SDC4, SLC34A2, and TMPRSS2.

According to the above, the partner DNA fragment and the target DNA fragment each includes a different sequence from a same gene selected from the group consisting of AR (e.g. ARV7), BCL2L1, BCL2-Like 11 (BIM or BCL2L11), BCOR, BIN1, BRAF, BRCA1, BRCA2, CASP2 (CASP-2), CD19, CD44, CXCR3, Cyclin D1 (CCND1), DMP1, CDH1, EGFR (e.g. EGFRvIII), ER (e.g. ESR1 or ESR2), EZH2, FAS, FGFR2, HRAS (H-RAS), IKZF1, KLF6, KRAS, MAP3K7, MCL1, MDM4, MET, MNK2, PIK3CD, PKM, RASGRP2, RON, RPS6KB, STAT3, TP53, TSC2, and VEGF.

According to the above, the DNA fragment joining event is selected from the group consisting of ACVR2A-AKT3, AFAP1-NTRK1, AFAP1-NTRK2, AFAP1-RET, AGAP3-BRAF, AGBL4-NTRK2, AGGF1-RAF1, AKAP13-NTRK3, AKAP13-RET, AKAP9-BRAF, AKT3-P2RX5, AKT3-PTPRR, AMOTL2-NTRK1, APIP-FGFR2, ARGLU1-NTRK1, ARHGEF11-NTRK1, ARHGEF2-NTRK1, ATG7-RAF1, ATP1B-NTRK1, AXL-MBIP, BAG4-FGFR1, BAIAP2L1-BRAF, BAIAP2L1-MET, BCAN-NTRK1, BCL6-RAF1, BCR-ABL, BCR-FGFR1, BCR-JAK2, BCR-NTRK2, BCR-RET, BRD3-NUTM1, BRD4-NUTM1, BTBD1-NTRK3, CAPZA2-MET, CBR4-ERBB4, CCDC6-BRAF, CCDC6-RET, CCDC6-ROS1, CD74-NRG1, CD74-NRG2, CD74-NTRK1, CD74-ROS1, CDK12-ERBB2, CDK5RAP2-BRAF, CEL-NTRK1, CEP170-AKT3, CHTOP-NTRK1, CLCN6-RAF1, CLIP1-ALK, CLIP1-ROS1, CLIP2-BRAF, CLIP2-MET, CLTC-ALK, CLTC-ROS1, CNTRL-KIT, COL25A1-ALK, COL25A1-FGFR2, COX5A-NTRK3, CPD-ERBB2, CTRC-NTRK1, CUX1-BRAF, CUX1-FGFR1, CUX1-RET, DCTN1-ALK, DCTN1-MET, DLG1-NTRK3, DNAJC8-ERBB2, EIF3E-RSPO2, EML1-NTRK2, EML4-ALK, EML4-BRAF, EML4-NTRK3, EML4-RET, EPHB2-NTRK1, EPS15-BRAF, EPS15-MET, EPS15-NTRK1, ERBB2-CDK12, ERBB2-CFB, ERBB2-CNIH4, ERBB2-CTTN, ERBB2-DNAJC7, ERBB2-ENO1, ERBB2-FCGRT, ERBB2-FKBP10, ERBB2-GRB7, ERBB2-GSE1, ERBB2-GTF2E2/SMIM18, ERBB2-IKZF3, ERBB2-KRT20, ERBB2-KRT39, ERBB2-KRTAP1-4, ERBB2-LMNTD1, ERBB2-LTBP4, ERBB2-MAD2L2, ERBB2-MED1, ERBB2-PARN, ERBB2-PGAP3, ERBB2-POLD4, ERBB2-PPP1R1B, ERBB2-PRDX4, ERBB2-PSMB3, ERBB2-SHKBP1, ERBB2-SLC39A11, ERBB2-SPTBN2, ERBB2-SRCIN1, ERBB2-TADA2A, ERBB2-TATDN1, ERBB2-XBP1, ERBB2-ZAN, ERBB4-AKAP6, ERBB4-FUS, ERBB4-IKZF2, ERBB4-STK11IP, ERC1-BRAF, ERC1-RET, ERC1-ROS1, ESRP1-RAF1, ESR1-CCDC170, ETV6-FGFR3, ETV6-NTRK2, ETV6-NTRK3, ETV6-PDGFRB, ETV6-PRDM16, EZR-ERBB4, EZR-ROS1, FAM131B-BRAF, FAT1-NTRK3, FGFR2-BICC1, FGFR2-TACC3, FGFR3-TACC3, FIP1L1-PDGFRA, FN1-ALK, FN1-ERBB4, FN1-FGFR1, FNDC3B-PIK3CA, FRY-NTRK3, GKAP1-NTRK2, GOLGA4-RAF1, GON4L-NTRK1, GOPC-ROS1, GRHL2-RSPO2, GRIPAP-NTRK1, GTF2IRD1-ALK, HACL1-RAF1, HIP1-ALK, HNRNPA2B1-NTRK3, IKZF2-ERBB4, IQSEC1-RAF1, IRF2BP2-NTRK1, KANK1-NTRK2, KCTD16-NTRK2, KCTD8-NTRK2, KHDRBS1-NTRK3, KIAA1549-BRAF, KIF5B-ALK, KIF5B-RET, KIF5B-ERBB4, KIT-ANKRD11, KIT-PDGFRA, KIT-SLC4A4, KMT2A-AFF1, KMT2A-CREBBP, KMT2A-DAB2IP, KMT2A-ELL, KMT2A-EPS15, KMT2A-MLLT1, KMT2A-MLLT10, KMT2A-MLLT11, KMT2A-MLLT3, KMT2A-MLLT4, KMT2A-SEP6, KMT2A-SEP9, KTN1-ALK, KTN1-RET, LIPI-NTRK1, LMNA-ALK, LMNA-NTRK1, LMNA-RAF1, LRRC71-NTRK1, LRRFIP1-FGFR1, LRRFIP1-MET, LYN-NTRK3, MAGI3-AKT3, MBNL1-RAF1, MEF2D-NTRK1, MET-MET, MIR548F1-NTRK1, MKRN1-BRAF, MPRIP-ALK, MPRIP-NTRK1, MPRIP-RAF1, MPRIP-RET, MRPL24-NTRK1, MSN-ALK, MSN-ROS1, MTSS1-ERBB2, MUC2-NTRK2, MYH9-ALK, MYO5A-NTRK3, MYO5A-ROS1, NACC2-NTRK2, NAV1-NTRK2, NBPF20-NTRK2, NCOA4-RET, NFASC-NTRK1, NOS1AP-NTRK1, NOS1AP-NTRK2, NRG2-CYSTM1, NRG2-UBE2D2, NRIP1-RSPO2, P2RY8-NTRK1, PAIP1-NTRK2, PAN3-NTRK2, PAPD7-RAF1, PDE4DIP-NTRK1, PEAR1-NTRK1, PHF20-NTRK1, PICALM-BRAF, PICALM-RET, PLEKHA6-NTRK1, PML-RARA, PPFIBP1-ALK, PPFIBP1-MET, PPFIBP1-ROS1, PPL-NTRK1, PRDX1-NTRK1, PRKAR1A-ALK, PRKAR1A-RET, PRKAR1B-ALK, PRKAR1B-BRAF, PRKAR2A-NTRK2, PRPSAP1-NTRK3, PTPRZ1-MET, QKI-NTRK2, QKI-RAF1, RAC1-AKT3, RAF1-ACTR2, RAF1-AGGF1, RAF1-DAZL, RAF1-ESRP1, RAF1-PHC3, RAF1-TMEM40, RAF1-TRAK1, RAF1-ZPR1, RALGPS2-NTRK3, RANBP2-ALK, RANBP2-FGFR1, RBPMS-NTRK3, RFWD2-NTRK1, RNF213-ALK, RNF213-NTRK1, RRBP1-ALK, RRBP1-RET, SATB1-ALK, SATB1-RET, SCAF11-PDGFRA, SCP2-NTRK1, SCYL3-NTRK1, SDC4-NRG1, SDC4-ROS1, SEC31A-ALK, SHC1-ERBB2, SIL1-NRG2, SLC34A2-MET, SLC34A2-ROS1, SLC45A3-BRAF, SLC45A3-ERG, SLC45A3-FGFR2, SLMAP-NTRK2, SND1-BRAF, SPECC1L-NTRK2, SPECC1L-NTRK3, SPTBN1-ALK, SQSTM1-ALK, SQSTM1-FGFR1, SQSTM1-NTRK1, SQSTM1-NTRK2, SQSTM1-NTRK3, SRGAP3-RAF1, SRGAP3-SRGAP3-RAF1, SSBP2-NTRK1, STRN-ALK, STRN-NTRK2, STRN-NTRK3, STRN3-BRAF, STRN3-NTRK1, STRN3-NTRK2, STRN3-NTRK3, TBC1D2-NTRK2, TBL1XR1-NRG1, TBL1XR1-PIK3CA, TBL1XR1-RET, TFG-ALK, TFG-MET, TFG-NTRK1, TFG-NTRK3, TFG-RET, TFG-ROS1, TIMP3-ALK, TIMP3-NTRK1, TKT-ERBB2, TLE4-NTRK2, TMEM106B-BRAF, TMEM106B-ROS1, TMPRSS2-ERG, TMPRSS2-ETV1, TMPRSS2-ETV4, TMPRSS2-ETV5, TNS3-NTRK2, TP53-NTRK1, TPM3-ALK, TPM3-NTRK1, TPM3-ROS1, TPM4-ALK, TPM4-NTRK3, TPR-ALK, TPR-BRAF, TPR-FGFR1, TPR-MET, TPR-NTRK1, TRAF2-NTRK2, TRAK1-RAF1, TRIM24-BRAF, TRIM24-FGFR1, TRIM24-NTRK2, TRIM24-RET, TRIM33-RET, TRIM33-NTRK1, TRIM4-BRAF, TRIM4-MET, TRIM63-NTRK1, UBE2R2-NTRK3, UFD1-NTRK2, USP13-PIK3CA, VANGL2-NTRK1, VCAN-NTRK2, VCL-ALK, VCL-NTRK2, VIM-NTRK3, VPS18-NTRK3, WHSC1L1-FGFR1, WHSC1L1-NUTM1, WIPF2-ERBB2, WNK2-NTRK2, ZBTB7B-NTRK1, and ZNF710-NTRK3 mutations.

According to the above, the third DNA fragment includes a sequence of a partner gene or a target gene.

According to the above, the DNA is amplified with the gene-specific primer first and subsequently with a universal primer to obtain the target nucleic acid in step (b) of the method.

According to the above, the signals are selected from the group consisting of dyes, chemiluminescent dyes, fluorescent molecules, radioisotopes, spin labels, enzymes, haptens, quantum dots, beads, aminohexyls, and pyrenes.

In one aspect, the present disclosure provides a method for distinguishing an alternative splicing event, and the method comprises steps of:
(a) probing a target nucleic acid with a split probe comprising
(i) a first split probe being complementary to the 3′ end of a partner DNA fragment, a second split probe being complementary to the 5′ end of a target DNA fragment, and/or a third split probe being complementary to a third DNA fragment, wherein a gap of the first and second split probes targeting sites on the target nucleic acid is within 0-80 bp; or
(ii) a first split probe being complementary to the 5′ end of a partner DNA fragment, a second split probe being complementary to the 3′ end of a target DNA fragment, and/or a third split probe being complementary to a third DNA fragment, wherein a gap of the first and second split probes targeting sites on the target nucleic acid is within 0-80 bp;
(b) detecting a signal that reflects a binding between the split probe and the target nucleic acid;
(c) determining
(i) the partner DNA fragment as an upstream DNA fragment and/or the target DNA fragment as a downstream DNA fragment through confirming the signal based on the first split probe binding to the 3′ end of the partner DNA fragment and/or the second split probe binding to the 5′ end of the target DNA fragment;
(ii) the partner DNA fragment as a downstream DNA fragment and/or the target DNA fragment as an upstream DNA fragment through confirming the signal based on the first split probe binding to the 5′ end of the partner DNA fragment and/or the second split probe binding to the 3′ end of the target DNA fragment; or
(iii) the third DNA fragment is joined with the partner DNA fragment and the target DNA fragment through confirming the signal based on the third split probe binding to the third DNA fragment; and
(d) comparing whether a length of the target nucleic acid is identical to a reference sequence of constitutive splicing.

According to the above, the target nucleic acid is amplified by a set of oligonucleotides.

According to the above, the target nucleic acid is amplified by multiplex PCR with at least two pairs of a gene-specific primers.

According to the above, the method further includes a step (e) for reconfirming by an independent PCR.

According to the above, at least two pairs of the gene-specific primers are designed to obtain the target nucleic acid from the partner DNA fragment as an upstream DNA fragment.

According to the above, at least two pairs of the gene-specific primers are designed to obtain the target nucleic acid from the partner DNA fragment as a downstream DNA fragment.

According to the above, at least one of the gene-specific primers targets a DNA fragment joining boundary.

According to the above, the gene-specific primer targets within a distance of 0-80 bp from a DNA fragment joining boundary.

According to the above, a product of multiplex PCR is amplified subsequently with a universal primer to obtain the target nucleic acid.

According to the above, a distance between the first and second split probes targeting site and a DNA fragment joining boundary is within 0-40 bp.

According to the above, a length of the split probe is 10-60 bp.

According to the above, the target nucleic acid is probed with a split probe and a single probe targeting a DNA fragment joining boundary in step (a) of the method.

According to the above, the partner DNA fragment and the target DNA fragment each includes a different sequence of a same gene selected from the group consisting of AR, BCL2L1, BCL2L11, BCOR, BIN1, BRAF, BRCA1, BRCA2, CASP2, CD19, CD44, CXCR3, CCND1, DMP1, CDH1, EGFR, ER, EZH2, FAS, FGFR2, HRAS, IKZF1, KLF6, KRAS, MAP3K7, MCL1, MDM4, MET, MNK2, PIK3CD, PKM, RASGRP2, RON, RPS6KB, STAT3, TP53, TSC2, and VEGF.

According to the above, the alternative splicing event is BCR-ABL mutation.

According to the above, the third DNA fragment includes a sequence of a partner gene or a target gene.

According to the above, the signal is selected from the group consisting of dyes, chemiluminescent dyes, fluorescent molecules, radioisotopes, spin labels, enzymes, haptens, quantum dots, beads, aminohexyls, and pyrenes.

In another aspect, the present disclosure also provides a method for treating a subject, and the method includes steps of
(a) determining whether a subject is at risk of cancer or genotype, including detecting a DNA fragment joining event by the method as described in the preceding paragraphs and/or distinguishing an alternative splicing event by the method as described in the preceding paragraphs of the sample from the subject; and
(b) administering
(i) a therapeutically effective amount of a siRNA targeting the DNA fragment joining event and/or the alternative splicing event;
(ii) a therapeutically effective amount of an inhibitor of a fusion protein encoded by the DNA fragment joining event and/or the alternative splicing event;
(iii) a therapeutically effective amount of an agent that inhibits a fusion protein encoded by the DNA fragment joining event and/or the alternative splicing event;
(iv) a therapeutically effective amount of an anticancer agent selected from the group consisting of cytokines, apoptosis-inducing agents, anti-angiogenic agents, chemotherapeutic agents, radio-therapeutic agents, and anticancer immunotoxins; or
(v) providing a targeted genome editing procedure within cells of the subject.

According to the above, the DNA fragment joining event and/or the alternative splicing event presents with a sequence of a partner gene selected from the group consisting of ACVR2A, AFAP1, AFF1, AGAP3, AGBL4, AGGF1, AKAP13, AKAP6, AKAP9, AMOTL2, ANKRD11, APIP, ARGLU1, ARHGEF11, ARHGEF2, ATG7, ATP1B, BAG4, BAIAP2L1, BCAN, BCL6, BCR, BICC1, BRD3, BRD4, BTBD1, CAPZA2, CBR4, CCDC170, CCDC6, CD74, CDK12, CDK5RAP2, CEL, CEP170, CFB, CHTOP, CLCN6, CLIP1, CLIP2, CLTC, CNIH4, CNTRL, COL25A1, COX5A, CPD, CREBBP, CTRC, CTTN, CUX1, CYSTM1, DAB2IP, DAZL, DCTN1, DLG1, DNAJC7, DNAJC8, EIF3E, ELL, EML1, EML4, ENO1, EPHB2, EPS15, ERC1, ESRP1, ETV6, EZR, FAM131B, FAT1, FCGRT, FGFR1, FGFR3, FIP1L1, FKBP10, FN1, FNDC3B, FRY, FUS, GKAP1, GOLGA4, GON4L, GOPC, GRB7, GRHL2, GRIPAP, GSE1, GTF2E2, GTF2IRD1, HACL1, HIP1, HNRNPA2B1, IKZF2, IKZF3, IQSEC1, IRF2BP2, JAK2, KANK1, KCTD16, KCTD8, KHDRBS1, KIAA1549, KIF5B, KRT20, KRT39, KRTAP1-4, KTN1, LIPI, LMNA, LMNTD1, LRRC71, LRRFIP1, LTBP4, LYN, MAD2L2, MAGI3, MBIP, MBNL1, MED1, MEF2D, MET, MIR548F1, MKRN1, MLLT1, MLLT10, MLLT11, MLLT3, MLLT4, MPRIP, MRPL24, MSN, MTSS1, MUC2, MYH9, MYO5A, NACC2, NAV1, NBPF20, NCOA4, NFASC, NOS1AP, NRG1, NRIP1, NTRK1, NTRK2, NTRK3, P2RX5, P2RY8, PAIP1, PAN3, PAPD7, PARN, PDE4DIP, PDGFRA, PDGFRB, PEAR1, PGAP3, PHC3, PHF20, PICALM, PLEKHA6, PML, POLD4, PPFIBP1, PPL, PPP1R1B, PRDM16, PRDX1, PRDX4, PRKAR1A, PRKAR1B, PRKAR2A, PRPSAP1, PSMB3, PTPRR, PTPRZ1, QKI, RAC1, RALGPS2, RANBP2, RBPMS, RET, RFWD2, RNF213, ROS1, RRBP1, SATB1, SCAF11, SCP2, SCYL3, SDC4, SEC31A, SEP6, SEP9, SHC1, SHKBP1, SIL1, SLC34A2, SLC39A11, SLC45A3, SLC4A4, SLMAP, SMIM18, SND1, SPECC1L, SPTBN1, SPTBN2, SQSTM1, SRCIN1, SRGAP3, SSBP2, STK11IP, STRN, STRN3, TACC3, TADA2A, TATDN1, TBC1D2, TBL1XR1, TFG, TIMP3, TKT, TLE4, TMEM106B, TMEM40, TMPRSS2, TNS3, TP53, TPM3, TPM4, TPR, TRAF2, TRAK1, TRIM24, TRIM33, TRIM4, TRIM63, UBE2D2, UBE2R2, UFD1, USP13, VANGL2, VCAN, VCL, VIM, VPS18, WHSC1L1, WIPF2, WNK2, XBP1, ZAN, ZBTB7B, ZNF710, and ZPR1.

According to the above, the DNA fragment joining event presents with a sequence of a target gene selected from the group consisting of ABL, AKT3, ALK, AXL, BCR, BRAF, CD74, ERBB2, ERBB4, ERG, ESR1, ETV1, ETV4, ETV5, ETV6, EZR, FGFR1, FGFR2, FGFR3, KIT, KMT2A, MET, NRG1, NRG2, NTRK1, NTRK2, NTRK3, NUTM1, PDGFRA, PDGFRB, PIK3CA, RAF1, RARA, RET, ROS1, RSPO2, SDC4, SLC34A2, and TMPRSS2.

According to the above, the alternative splicing event presents with a different sequence of a same gene selected from the group consisting of AR, BCL2L1, BCL2L11, BCOR, BIN1, BRAF, BRCA1, BRCA2, CASP2, CD19, CD44, CXCR3, CCND1, DMP1, CDH1, EGFR, ER, EZH2, FAS, FGFR2, HRAS, IKZF1, KLF6, KRAS, MAP3K7, MCL1, MDM4, MET, MNK2, PIK3CD, PKM, RASGRP2, RON, RPS6KB, STAT3, TP53, TSC2, and VEGF.

According to the above, the DNA fragment joining or the alternative splicing event is BCR-ABL mutation.

According to the above, the alternative splicing event is selected from the group consisting of constitutive splicing, exon skipping, intron retention, mutually exclusive exons, and alternative 5′ or 3′ splice site.

According to the above, the cancer is selected from the group consisting of carcinoma, sarcoma, lymphoma, leukemia, and myeloma.

According to the above, the cancer is selected from the group consisting of brain cancer, breast cancer, colon cancer, endocrine gland cancer, esophageal cancer, female reproductive organ cancer, head and neck cancer, hepatobiliary system cancer, kidney cancer, lung cancer, mesenchymal cell neoplasm, prostate cancer, skin cancer, stomach cancer, tumor of the exocrine pancreas, and urinary system cancer.

In another aspect, the present disclosure also provides a kit for detecting a sample with a DNA fragment joining event and/or an alternative splicing event, and the kit includes:
(a) a set of oligonucleotides;
(b) a split probe, having:
(i) a first split probe being complementary to the 3′ end of a partner DNA fragment, a second split probe being complementary to the 5′ end of a target DNA fragment, and/or a third split probe being complementary to a third DNA fragment, wherein a gap of the first and second split probes targeting sites on a target nucleic acid is within 0-80 bp; or
(ii) a first split probe being complementary to the 5′ end of a partner DNA fragment, a second split probe being complementary to the 3′ end of a target DNA fragment, and/or a third split probe being complementary to a third DNA fragment, wherein a gap of the split probes targeting sites on a target nucleic acid is within 0-80 bp; and
(c) a probe hybridization assay for detecting a split probe hybridization signals, which includes dyes, chemiluminescent dyes, fluorescent molecules, radioisotopes, spin labels, enzymes, haptens, quantum dots, beads, aminohexyls, and pyrenes.

According to the above, the set of oligonucleotides is a gene-specific primer or a gene-specific probe.

According to the above, the kit comprises at least two pairs of a gene-specific primers.

According to the above, the gene-specific primer is designed to obtain the target nucleic acid from the partner DNA fragment as an upstream DNA fragment.

According to the above, the gene-specific primer is designed to obtain the target nucleic acid from the partner DNA fragment as a downstream DNA fragment.

According to the above, the kit further includes a universal primer.

According to the above, at least one of the gene-specific primers targets a DNA fragment joining boundary.

According to the above, the gene-specific primer targets within a distance of 0-80 bp from a DNA fragment joining boundary.

According to the above, the first split probe or the second split probe targets within a distance of 0-40 bp from a DNA fragment joining boundary.

According to the above, the first split probe is selected from the group consisting of SEQ ID Nos: 32, 35, and any complementary sequence thereof.

According to the above, the second split probe is selected from the group consisting of SEQ ID Nos: 33, 36, and any complementary sequence thereof.

According to the above, the third split probe is selected from the group consisting of SEQ ID Nos: 32, 33, 35, 36, and any complementary sequence thereof.

In another aspect, the disclosure provides the kit including a length of the split probe is 10-60 bp.

According to the above, the kit further includes a single probe targeting a DNA fragment joining boundary.

According to the above, the first split probe is complementary to a sequence of a partner gene selected from the group consisting of ACVR2A, AFAP1, AFF1, AGAP3, AGBL4, AGGF1, AKAP13, AKAP6, AKAP9, AMOTL2, ANKRD11, APIP, ARGLU1, ARHGEF11, ARHGEF2, ATG7, ATP1B, BAG4, BAIAP2L1, BCAN, BCL6, BCR, BICC1, BRD3, BRD4, BTBD1, CAPZA2, CBR4, CCDC170, CCDC6, CD74, CDK12, CDK5RAP2, CEL, CEP170, CFB, CHTOP, CLCN6, CLIP1, CLIP2, CLTC, CNIH4, CNTRL, COL25A1, COX5A, CPD, CREBBP, CTRC, CTTN, CUX1, CYSTM1, DAB2IP, DAZL, DCTN1, DLG1, DNAJC7, DNAJC8, EIF3E, ELL, EML1, EML4, ENO1, EPHB2, EPS15, ERC1, ESRP1, ETV6, EZR, FAM131B, FAT1, FCGRT, FGFR1, FGFR3, FIP1L1, FKBP10, FN1, FNDC3B, FRY, FUS, GKAP1, GOLGA4, GON4L, GOPC, GRB7, GRHL2, GRIPAP, GSE1, GTF2E2, GTF2IRD1, HACL1, HIP1, HNRNPA2B1, IKZF2, IKZF3, IQSEC1, IRF2BP2, JAK2, KANK1, KCTD16, KCTD8, KHDRBS1, KIAA1549, KIF5B, KRT20, KRT39, KRTAP1-4, KTN1, LIPI, LMNA, LMNTD1, LRRC71, LRRFIP1, LTBP4, LYN, MAD2L2, MAGI3, MBIP, MBNL1, MED1, MEF2D, MET, MIR548F1, MKRN1, MLLT1, MLLT10, MLLT11, MLLT3, MLLT4, MPRIP, MRPL24, MSN, MTSS1, MUC2, MYH9, MYO5A, NACC2, NAV1, NBPF20, NCOA4, NFASC, NOS1AP, NRG1, NRIP1, NTRK1, NTRK2, NTRK3, P2RX5, P2RY8, PAIP1, PAN3, PAPD7, PARN, PDE4DIP, PDGFRA, PDGFRB, PEAR1, PGAP3, PHC3, PHF20, PICALM, PLEKHA6, PML, POLD4, PPFIBP1, PPL, PPP1R1B, PRDM16, PRDX1, PRDX4, PRKAR1A, PRKAR1B, PRKAR2A, PRPSAP1, PSMB3, PTPRR, PTPRZ1, QKI, RAC1, RALGPS2, RANBP2, RBPMS, RET, RFWD2, RNF213, ROS1, RRBP1, SATB1, SCAF11, SCP2, SCYL3, SDC4, SEC31A, SEP6, SEP9, SHC1, SHKBP1, SIL1, SLC34A2, SLC39A11, SLC45A3, SLC4A4, SLMAP, SMIM18, SND1, SPECC1L, SPTBN1, SPTBN2, SQSTM1, SRCIN1, SRGAP3, SSBP2, STK11IP, STRN, STRN3, TACC3, TADA2A, TATDN1, TBC1D2, TBL1XR1, TFG, TIMP3, TKT, TLE4, TMEM106B, TMEM40, TMPRSS2, TNS3, TP53, TPM3, TPM4, TPR, TRAF2, TRAK1, TRIM24, TRIM33, TRIM4, TRIM63, UBE2D2, UBE2R2, UFD1, USP13, VANGL2, VCAN, VCL, VIM, VPS18, WHSC1L1, WIPF2, WNK2, XBP1, ZAN, ZBTB7B, ZNF710, and ZPR1.

According to the above, the second split probe is complementary to a sequence of a target gene selected from the group consisting of ABL, AKT3, ALK, AXL, BCR, BRAF, CD74, ERBB2, ERBB4, ERG, ESR1, ETV1, ETV4, ETV5, ETV6, EZR, FGFR1, FGFR2, FGFR3, KIT, KMT2A, MET, NRG1, NRG2, NTRK1, NTRK2, NTRK3, NUTM1, PDGFRA, PDGFRB, PIK3CA, RAF1, RARA, RET, ROS1, RSPO2, SDC4, SLC34A2, and TMPRSS2.

According to the above, the first split probe and the second split probe are complementary to the partner DNA fragment and the target DNA fragment each including a different sequence of a same gene selected from the group consisting of AR, BCL2L1, BCL2L11, BCOR, BIN1, BRAF, BRCA1, BRCA2, CASP2, CD19, CD44, CXCR3, CCND1, DMP1, CDH1, EGFR, ER, EZH2, FAS, FGFR2, HRAS, IKZF1, KLF6, KRAS, MAP3K7, MCL1, MDM4, MET, MNK2, PIK3CD, PKM, RASGRP2, RON, RPS6KB, STAT3, TP53, TSC2, and VEGF.

According to the above, the DNA fragment joining event or the alternative splicing event is BCR-ABL mutation.

According to the above, the third split probe is complementary to the third DNA fragment having a sequence of a partner gene or a target gene.

### BRIEF DESCRIPTION OF DRAWINGS

The disclosure will be apparent to those skilled in the art from the following detailed description of the preferred embodiments, regarding the attached drawings, in which:

FIG. 1 is a schematic illustration of the method for detecting MET gene mutation according to one embodiment of the invention; the detection is based on a one-step PCR target-probe hybridization assay.

FIG. 2 is a schematic illustration of the method for detecting NTRK gene mutation according to one embodiment of the invention; the detection is based on a two-step PCR target-probe hybridization assay.

FIG. 3 is a schematic illustration of the method for detecting EGFR gene mutation according to one embodiment of the invention; the detection is based on a two-step PCR target-probe hybridization assay.

FIG. 4 shows an array of probe spots printed in a well of a plate; the numbers in the first line from the top and the first column from the left are presented as reference coordinates; the squares labeled 1-117 represent the spots of the split probes, the squares labeled IC001-IC009 represent the spots of control probes, and the squares labeled R144 represent the spots of an anchor probe.

FIG. 5 shows an ETV6-NTRK3 (Exon 5 and Exon 14) fusion-positive array of probe spots in a well of a plate according to one embodiment of the invention.

FIG. 6 shows an QKI-NTRK2 (Exon 6 and Exon 16) fusion-positive array of probe spots in a well of a plate according to one embodiment of the invention.

FIG. 7 shows an AFAP1-NTRK1 (Exon 4 and Exon 10) fusion-positive array of probe spots in a well of a plate according to one embodiment of the invention.

FIG. 8 shows an artificial novel PPL-NTRK3 (Exon 22 and Exon 14) fusion-positive array of probe spots in a well of a plate according to one embodiment of the invention.

FIG. 9 shows fusion-negative and fusion-positive arrays of probe spots in a well of control group (water, fusion-negative samples) and fusion group (PPL-NTRK3).

### DETAILED DESCRIPTION OF THE INVENTION

Unless defined otherwise, all technical and scientific terms used herein have the same meaning as commonly understood by a person skilled in the art to which this disclosure belongs.

### Definition

As used herein, the singular forms “a”, “an”, and “the” include plural referents unless the context dictates otherwise.

The term “DNA fragment joining” refers to rearrangement, translocation, tandem repeat, inversion, insertion, deletion, or other chimeric variation of DNA through breaking and rejoining of DNA fragments. DNA fragment joining occurs when a hybrid DNA fragment is generated from two or more DNA fragments that are normally separated. The term “DNA fragment joining” also refers to when a cDNA product is synthesized from an mRNA that followed an alternative splicing pathway.

The term “alternative splicing” refers to an event in which a primary transcript can be spliced into more than one isoform of mRNAs. Different categories of alternative splicing have been disclosed, including constitutive splicing, exon skipping, intron retention, mutually exclusive exons, and alternative 5′ or 3′ splice sites.

The term “target DNA fragment” refers to any nucleic acid molecule, polynucleotide sequence, or any fragment comprising a portion of a specific gene or genetic locus in the genomic DNA. The term “partner DNA fragment” refers to the fragment whose 3′ or 5′ sequence is joined to the 5′ or 3′ sequence of the “target DNA fragment.” The target DNA fragment or the partner DNA fragment includes an intact gene, an exon or intron, a regulatory sequence, or any region between genes. The DNA fragment at the 5′ end of the hybrid DNA fragment is referred to as “upstream DNA fragment”, and the DNA fragment at the 3′ end of the hybrid DNA fragment is referred to as “downstream DNA fragment.”

The hybrid DNA fragment has a “DNA fragment joining boundary” which is the region where one DNA fragment is joined to another DNA fragment. For example, the regions that the partner DNA fragment is joined to the target DNA fragment, or the regions that the partner DNA fragment is joined to another DNA fragment or fusion junction. The joining between two or more specific DNA fragments is further multiplied by the DNA fragment joining boundary.

Sometimes the target DNA fragment and the partner DNA fragment comprised of specific gene sequences are joined in aberrant combinations to cause gene fusion. The term “gene fusion” refers to a phenomenon where a first gene on a chromosome is fused to a second gene on the same or a different chromosome and a hybrid gene or a fusion gene thus forms. This phenomenon is also commonly referred to as “gene translocation” or “gene rearrangement.” For example, when an NTRK gene is one of the multiple fused genes, such gene fusion is called “NTRK gene fusion” or “NTRK fusion.”

The gene at the 5′ end of the fusion gene is referred to as the “5′ gene”, and the gene at the 3′ end of the fusion gene is referred to as the “3′ gene.” The fusion gene has a “fusion junction,” which is the site where the gene fuses. A fusion junction is located in a fusion region defined by a fusion sequence (also called a fusion junction sequence), which encompasses the sequence from the 5′ gene and the sequence from the 3′ gene. Different combinations of fusion genes lead to different “fusion types.” The fusion between two specific genes is further diversified by fusion junction since fusion junction may occur anywhere within the fusion genes. For example, the fusion between the first exon of a first gene and the second exon of a second gene is one fusion type, whereas the fusion between the third exon of the first gene and the first exon of the second gene is another fusion type.

Gene fusions may be detected by identifying a fusion junction in a DNA or in an RNA transcript of that DNA. As used herein, a “fusion type” refers to a unique fusion present in an RNA transcript. In other words, it is considered the same fusion type when the fusions between two specific genes occur at different sites within the same intronic region. For example, a fusion between exon 3 of gene A and exon 5 of gene B may have a DNA fusion region containing a small portion of the intron between exons 3 and 4 of gene A and a large portion of the intron between exons 4 and 5 of gene B. Alternatively, such fusion may have a DNA fusion region containing a large portion of the intron between exons 3 and 4 of gene A and a small portion of the intron between exons 4 and 5 of gene B. These two fusions, though having different DNA fusion junctions, are considered the same “fusion type” because the RNA transcripts generated from the two fusions are the same.

The term “a set of oligonucleotides” refers to a set of synthetic single-stranded oligonucleotides that can be used to enrich target genetic regions for sequencing. As used herein, the terms “gene-specific primer (pair),” “MET mutation-specific primer (pair),” “NTRK fusion-specific primer (pair),” or “EGFRvIII mutation-specific primer (pair)” refer to a DNA primer that is designed to amplify a target DNA including a DNA fragment joining boundary or a fusion junction. As used herein, the term “gene-specific probe” refers to a synthesized oligonucleotide probe (as baits) that is complementary to the target genetic sequence.

As used herein, the term “universal primer” refers to a DNA primer that is designed to amplify any DNA including the nucleotide sequence of the universal primer. The universal primers are used in pairs, including a universal forward primer and a universal reverse primer.

Unless defined otherwise, the term “split probe” refers to two or more synthetic single-stranded DNA oligonucleotides that can hybridize to the DNA fragment joining region that originates from the partner DNA fragment and the target DNA fragment and/or another DNA fragment.

Each of the genes described herein corresponds to a “gene name (or symbol)” listed in the NCBI gene database (https://www.ncbi.nlm.nih.gov/gene/). The NCBI gene database, therefore, is used to identify the sequence of a gene or synonyms of the gene name.

In the present disclosure, a method for detecting a DNA fragment joining event is provided. The method includes the steps of:
(a) obtaining a DNA or a DNA from an extracted RNA in a sample;
(b) enriching the DNA with a set of oligonucleotides to obtain a target amplified nucleic acid;
(c) probing the target amplified nucleic acid with a split probe comprising
(i) a first split probe that is complementary to the 3′ end of a partner DNA fragment, a second split probe that is complementary to the 5′ end of a target DNA fragment, and/or a third split probe that is complementary to an another DNA fragment, wherein a gap of the split probe targeting sites on the target amplified nucleic acid are within a distance of 0-80 bp from one another, or
(ii) a first split probe that is complementary to the 5′ end of a partner DNA fragment, a second split probe that is complementary to the 3′ end of a target DNA fragment, and/or a third split probe that is complementary to an another DNA fragment, wherein a gap of the split probe targeting sites on the target nucleic acid is within a distance of 0-80 bp from one another; and
(d) detecting a signal that reflects the binding between the split probe and the target nucleic acid.

In some embodiments, RNA is prepared from a biological sample. The biological sample may be any sample obtained from an animal and a human subject. Examples of the biological samples include a formalin-fixed paraffin-embedded (FFPE) tissue section, peripheral blood mononuclear cells (PBMCs), blood, plasma, or other cells or body fluids. In some embodiments, the biological sample originates from a cancer patient. In some embodiments, the biological sample originates from a carcinoma, a sarcoma, a lymphoma, a leukemia, or a myeloma. In some embodiments, the biological sample originates from a patient with brain cancer, breast cancer, colon cancer, endocrine gland cancer, esophageal cancer, female reproductive organ cancer, head and neck cancer, hepatobiliary system cancer, kidney cancer, lung cancer, mesenchymal cell neoplasm, prostate cancer, skin cancer, stomach cancer, tumor of the exocrine pancreas, and urinary system cancer.

Preparation of total RNA from the biological sample can be carried out by various methods known in the art. One typical procedure is RNA extraction with organic solvents such as phenol/chloroform and precipitation by centrifugation. There are also commercially available kits for RNA isolation or purification. Once the RNA is obtained, a reverse transcriptase is used along with four kinds of deoxyribonucleoside triphosphates (dNTP, including dATP, dCTP, dTTP, and dGTP) to generate cDNA from the template RNA, a process called reverse transcription. The reverse transcription may be conducted using the SuperScript cDNA synthesis kit (Cat No: 11754050, Invitrogen).

In some embodiments, the disclosed method further includes a step of determining:
(i) the partner DNA fragment as an upstream DNA fragment and/or the target DNA fragment as a downstream DNA fragment through confirming the signal based on the first split probe binding to the 3′ end of the partner DNA fragment and/or the second split probe binding to the 5′ end of the target DNA fragment;
(ii) the partner DNA fragment as a downstream DNA fragment and/or the target DNA fragment as an upstream DNA fragment through confirming the signal based on the first split probe binding to the 5′ end of the partner DNA fragment and/or the second split probe binding to the 3′ end of the target DNA fragment or
(iii) whether or not the third DNA fragment is joined with the partner DNA fragment and the target DNA fragment through confirming the signal based on the third split probe binding to the third DNA fragment and a result of the target nucleic acid from an independent PCR.

In the present disclosure, a method for distinguishing an alternative splicing event is also provided. The method includes the steps of:
(a) probing a target amplified nucleic acid with a split probe having:
(i) a first split probe that is complementary to the 3′ end of a partner DNA fragment, a second split probe that is complementary to the 5′ end of a target DNA fragment, and/or a third split probe that is complementary to an another DNA fragment, wherein a gap of the split probe targeting sites on the target nucleic acid are within a distance of 0-80 bp from one another or
(ii) a first split probe that is complementary to the 5′ end of a partner DNA fragment, a second split probe that is complementary to the 3′ end of a target DNA fragment, and/or a third split probe that is complementary to an another DNA fragment, wherein a gap of the split probe targeting sites on the target nucleic acid is within a distance of 0-80 bp from one another;
(b) detecting a signal that reflects the binding between the split probe and the target nucleic acid;
(c) determining
(i) the partner DNA fragment as an upstream DNA fragment and/or the target DNA fragment as a downstream DNA fragment through confirming the signals based on the first split probe binding to the 3′ end of the partner DNA fragment and/or the second split probe binding to the 5′ end of the target DNA fragment;
(ii) the partner DNA fragment as a downstream DNA fragment and/or the target DNA fragment as an upstream DNA fragment through confirming the signals based on the first split probe binding to the 5′ end of the partner DNA fragment and/or the second split probe binding to the 3′ end of the target DNA fragment, or
(iii) the third DNA fragment is joined with the partner DNA fragment and the target DNA fragment through confirming the signal based on the third split probe binding to the third DNA fragment; and
(d) comparing whether the length of the target nucleic acid is identical to a reference sequence of constitutive splicing.

In some embodiments, the set of oligonucleotides is a gene-specific primer or a gene-specific probe.

In some embodiments, the target amplified nucleic acid is amplified by the multiplex PCR with at least two pairs of the gene-specific primers.

In some embodiments, the method as described in the preceding paragraphs further includes a step of reconfirming by an independent PCR (e.g. Sanger PCR or qPCR).

In some embodiments, the DNA is amplified with a DNA polymerase and at least two pairs of a gene-specific primers to obtain a target amplified nucleic acid for probe detection. In some embodiments, the gene-specific primer is an NTRK fusion-specific primer, a MET mutation-specific primer, or an EGFRvIII mutation-specific primer. The amplification may be conducted using a multiplex PCR kit (Cat No: 206143, Qiagen) which includes a DNA polymerase. The gene-specific primer may be provided as a regent before use. In some embodiments, one NTRK fusion-specific primer is used to amplify each target nucleic acid. In some embodiments, two or more pairs of the NTRK fusion-specific primers are pooled together to amplify each target nucleic acid.

In some embodiments, two or more pairs of the gene-specific primers which consist of NTRK fusion-specific primer, MET mutation-specific primer, and EGFRvIII mutation-specific primer are pooled together to amplify each target nucleic acid.

In other embodiments, the gene-specific primer is partially pooled to form a plurality of pooled reagents, each of which contains at least one pair of the gene-specific primer. Thus, the number of the pooled reagent may be 1, 2, 3, 4, 5, 6, 7, 8, 9, 10, or more. In some preferred embodiments, over a hundred pairs of the NTRK fusion-specific primer are provided in four pooled reagents to be used in four multiplex amplification reactions. DNA amplification in this manner has been demonstrated to show significantly higher performance than a single multiplex amplification reaction carried out with all the gene-specific primers, probably due to decreased primer complexity.

In some preferred embodiments, the DNA is amplified first with at least two pairs of the NTRK fusion-specific primers and subsequently with a universal primer to obtain the target amplified nucleic acid. When the universal primer is utilized in the disclosed method, the gene-specific forward primer in each pair of the gene-specific primer further encompasses the nucleotide sequence of the universal forward primer in the pair of universal primers, and the gene-specific reverse primer in each pair of the gene-specific primer further encompasses the nucleotide sequence of the universal reverse primer in the pair of universal primers. The use of the universal primer can increase the ultimate yield of any possible amplified product, no matter which DNA fragment is to be detected or which gene-specific primer is used in the first round of amplification. An additional advantage of applying the universal primers is that when primers are to be modified to become detectable, only two or even one universal primer needs to be modified, for example, forming a linkage between one universal primer and a connector (such as biotin) so that a detectable molecule can be linked to the universal primer. Otherwise, all gene-specific primers have to be modified, which makes the primer modification process more complicated and costly.

In some preferred embodiments, the target amplified nucleic acid is mixed with the split probe so that a probe-bound product that reflects the bindings between the split probe and the target amplified nucleic acid can form through nucleic acid hybridization.

In some embodiments, because the split probe is specifically designed oriented on a specific sequence of the partner DNA fragment, the target DNA fragment, or the third DNA fragment, the exact DNA fragment joining event can be determined by detecting a signal of the first split probe, the second split probe or the third split probe from a particular probe-bound product.

In some embodiments, the length of each of the split probes is 10-60 bp. In some embodiments, the length of the target nucleic acid is no longer than 200 bp.

In some preferred embodiments, the DNA is amplified with at least two pairs of the gene-specific primers that are designed to obtain the target nucleic acid from which the partner DNA fragment as an upstream DNA fragment.

In some preferred embodiments, the DNA is amplified with at least two pairs of the gene-specific primers that are designed to obtain the target nucleic acid from which the partner DNA fragment as a downstream DNA fragment.

In some embodiments, the DNA fragment joining that can be detected includes, but is not limited to, a rearrangement, translocation, tandem repeat, inversion, insertion, deletion, or other chimeric variation events of the sample.

In some embodiments, the gene mutation that can be detected includes, but is not limited to, a fusion between a target gene that is selected from the group consisting of ABL, AKT3, ALK, AXL, BCR, BRAF, CD74, ERBB2, ERBB4, ERG, ESR1, ETV1, ETV4, ETV5, ETV6, EZR, FGFR1, FGFR2, FGFR3, KIT, KMT2A, MET, NRG1, NRG2, NTRK1, NTRK2, NTRK3, NUTM1, PDGFRA, PDGFRB, PIK3CA, RAF1, RARA, RET, ROS1, RSPO2, SDC4, SLC34A2, and TMPRSS2 and a partner gene that is selected from the group consisting of ACVR2A, AFAP1, AFF1, AGAP3, AGBL4, AGGF1, AKAP13, AKAP6, AKAP9, AMOTL2, ANKRD11, APIP, ARGLU1, ARHGEF11, ARHGEF2, ATG7, ATP1B, BAG4, BAIAP2L1, BCAN, BCL6, BCR, BICC1, BRD3, BRD4, BTBD1, CAPZA2, CBR4, CCDC170, CCDC6, CD74, CDK12, CDK5RAP2, CEL, CEP170, CFB, CHTOP, CLCN6, CLIP1, CLIP2, CLTC, CNIH4, CNTRL, COL25A1, COX5A, CPD, CREBBP, CTRC, CTTN, CUX1, CYSTM1, DAB2IP, DAZL, DCTN1, DLG1, DNAJC7, DNAJC8, EIF3E, ELL, EML1, EML4, ENO1, EPHB2, EPS15, ERC1, ESRP1, ETV6, EZR, FAM131B, FAT1, FCGRT, FGFR1, FGFR3, FIP1L1, FKBP10, FN1, FNDC3B, FRY, FUS, GKAP1, GOLGA4, GON4L, GOPC, GRB7, GRHL2, GRIPAP, GSE1, GTF2IRD1, HACL1, HIP1, HNRNPA2B1, IKZF2, IKZF3, IQSEC1, IRF2BP2, JAK2, KANK1, KCTD16, KCTD8, KHDRBS1, KIAA1549, KIF5B, KRT20, KRT39, KRTAP1-4, KTN1, LIPI, LMNA, LMNTD1, LRRC71, LRRFIP1, LTBP4, LYN, MAD2L2, MAGI3, MBIP, MBNL1, MED1, MEF2D, MET, MIR548F1, MKRN1, MLLT1, MLLT10, MLLT11, MLLT3, MLLT4, MPRIP, MRPL24, MSN, MTSS1, MUC2, MYH9, MYO5A, NACC2, NAV1, NBPF20, NCOA4, NFASC, NOS1AP, NRG1, NRIP1, NTRK1, NTRK2, NTRK3, P2RX5, P2RY8, PAIP1, PAN3, PAPD7, PARN, PDE4DIP, PDGFRA, PDGFRB, PEAR1, PGAP3, PHC3, PHF20, PICALM, PLEKHA6, PML, POLD4, PPFIBP1, PPL, PPP1R1B, PRDM16, PRDX1, PRDX4, PRKAR1A, PRKAR1B, PRKAR2A, PRPSAP1, PSMB3, PTPRR, PTPRZ1, QKI, RAC1, RALGPS2, RANBP2, RBPMS, RET, RFWD2, RNF213, ROS1, RRBP1, SATB1, SCAF11, SCP2, SCYL3, SDC4, SEC31A, SEP6, SEP9, SHC1, SHKBP1, SIL1, SLC34A2, SLC39A11, SLC45A3, SLC4A4, SLMAP, SND1, SPECC1L, SPTBN1, SPTBN2, SQSTM1, SRCIN1, SRGAP3, SSBP2, STK11IP, STRN, STRN3, TACC3, TADA2A, TATDN1, TBC1D2, TBL1XR1, TFG, TIMP3, TKT, TLE4, TMEM106B, TMEM40, TMPRSS2, TNS3, TP53, TPM3, TPM4, TPR, TRAF2, TRAK1, TRIM24, TRIM33, TRIM4, TRIM63, UBE2D2, UBE2R2, UFD1, USP13, VANGL2, VCAN, VCL, VIM, VPS18, WHSC1L1, WIPF2, WNK2, XBP1, ZAN, ZBTB7B, ZNF710, and ZPR1.

In some embodiments, the partner DNA fragment includes a sequence of a partner gene selected from the group consisting of ACVR2A, AFAP1, AFF1, AGAP3, AGBL4, AGGF1, AKAP13, AKAP6, AKAP9, AMOTL2, ANKRD11, APIP, ARGLU1, ARHGEF11, ARHGEF2, ATG7, ATP1B, BAG4, BAIAP2L1, BCAN, BCL6, BCR, BICC1, BRD3, BRD4, BTBD1, CAPZA2, CBR4, CCDC170, CCDC6, CD74, CDK12, CDK5RAP2, CEL, CEP170, CFB, CHTOP, CLCN6, CLIP1, CLIP2, CLTC, CNIH4, CNTRL, COL25A1, COX5A, CPD, CREBBP, CTRC, CTTN, CUX1, CYSTM1, DAB2IP, DAZL, DCTN1, DLG1, DNAJC7, DNAJC8, EIF3E, ELL, EML1, EML4, ENO1, EPHB2, EPS15, ERC1, ESRP1, ETV6, EZR, FAM131B, FAT1, FCGRT, FGFR1, FGFR3, FIP1L1, FKBP10, FN1, FNDC3B, FRY, FUS, GKAP1, GOLGA4, GON4L, GOPC, GRB7, GRHL2, GRIPAP, GSE1, GTF2IRD1, HACL1, HIP1, HNRNPA2B1, IKZF2, IKZF3, IQSEC1, IRF2BP2, JAK2, KANK1, KCTD16, KCTD8, KHDRBS1, KIAA1549, KIF5B, KRT20, KRT39, KRTAP1-4, KTN1, LIPI, LMNA, LMNTD1, LRRC71, LRRFIP1, LTBP4, LYN, MAD2L2, MAGI3, MBIP, MBNL1, MED1, MEF2D, MET, MIR548F1, MKRN1, MLLT1, MLLT10, MLLT11, MLLT3, MLLT4, MPRIP, MRPL24, MSN, MTSS1, MUC2, MYH9, MYO5A, NACC2, NAV1, NBPF20, NCOA4, NFASC, NOS1AP, NRG1, NRIP1, NTRK1, NTRK2, NTRK3, P2RX5, P2RY8, PAIP1, PAN3, PAPD7, PARN, PDE4DIP, PDGFRA, PDGFRB, PEAR1, PGAP3, PHC3, PHF20, PICALM, PLEKHA6, PML, POLD4, PPFIBP1, PPL, PPP1R1B, PRDM16, PRDX1, PRDX4, PRKAR1A, PRKAR1B, PRKAR2A, PRPSAP1, PSMB3, PTPRR, PTPRZ1, QKI, RAC1, RALGPS2, RANBP2, RBPMS, RET, RFWD2, RNF213, ROS1, RRBP1, SATB1, SCAF11, SCP2, SCYL3, SDC4, SEC31A, SEP6, SEP9, SHC1, SHKBP1, SIL1, SLC34A2, SLC39A11, SLC45A3, SLC4A4, SLMAP, SND1, SPECC1L, SPTBN1, SPTBN2, SQSTM1, SRCIN1, SRGAP3, SSBP2, STK11IP, STRN, STRN3, TACC3, TADA2A, TATDN1, TBC1D2, TBL1XR1, TFG, TIMP3, TKT, TLE4, TMEM106B, TMEM40, TMPRSS2, TNS3, TP53, TPM3, TPM4, TPR, TRAF2, TRAK1, TRIM24, TRIM33, TRIM4, TRIM63, UBE2D2, UBE2R2, UFD1, USP13, VANGL2, VCAN, VCL, VIM, VPS18, WHSC1L1, WIPF2, WNK2, XBP1, ZAN, ZBTB7B, ZNF710, and ZPR1.

In some embodiments, the target DNA fragment includes a sequence of a target gene selected from the group consisting of ABL, AKT3, ALK, AXL, BCR, BRAF, CD74, ERBB2, ERBB4, ERG, ESR1, ETV1, ETV4, ETVS, ETV6, EZR, FGFR1, FGFR2, FGFR3, KIT, KMT2A, MET, NRG1, NRG2, NTRK1, NTRK2, NTRK3, NUTM1, PDGFRA, PDGFRB, PIK3CA, RAF1, RARA, RET, ROS1, RSPO2, SDC4, SLC34A2, and TMPRSS2.

In some embodiments, the third DNA fragment includes a sequence of the partner gene or the target gene.

In some embodiment, the detectable gene mutation types include, but is not limited to, ACVR2A-AKT3, AFAP1-NTRK1, AFAP1-NTRK2, AFAP1-RET, AGAP3-BRAF, AGBL4-NTRK2, AGGF1-RAF1, AKAP13-NTRK3, AKAP13-RET, AKAP9-BRAF, AKT3-P2RX5, AKT3-PTPRR, AMOTL2-NTRK1, APIP-FGFR2, ARGLU1-NTRK1, ARHGEF11-NTRK1, ARHGEF2-NTRK1, ATG7-RAF1, ATP1B-NTRK1, AXL-MBIP, BAG4-FGFR1, BAIAP2L1-BRAF, BAIAP2L1-MET, BCAN-NTRK1, BCL6-RAF1, BCR-ABL, BCR-FGFR1, BCR-JAK2, BCR-NTRK2, BCR-RET, BRD3-NUTM1, BRD4-NUTM1, BTBD1-NTRK3, CAPZA2-MET, CBR4-ERBB4, CCDC6-BRAF, CCDC6-RET, CCDC6-ROS1, CD74-NRG1, CD74-NRG2, CD74-NTRK1, CD74-ROS1, CDK12-ERBB2, CDK5RAP2-BRAF, CEL-NTRK1, CEP170-AKT3, CHTOP-NTRK1, CLCN6-RAF1, CLIP1-ALK, CLIP1-ROS1, CLIP2-BRAF, CLIP2-MET, CLTC-ALK, CLTC-ROS1, CNTRL-KIT, COL25A1-ALK, COL25A1-FGFR2, COX5A-NTRK3, CPD-ERBB2, CTRC-NTRK1, CUX1-BRAF, CUX1-FGFR1, CUX1-RET, DCTN1-ALK, DCTN1-MET, DLG1-NTRK3, DNAJC8-ERBB2, EIF3E-RSPO2, EML1-NTRK2, EML4-ALK, EML4-BRAF, EML4-NTRK3, EML4-RET, EPHB2-NTRK1, EPS15-BRAF, EPS15-MET, EPS15-NTRK1, ERBB2-CDK12, ERBB2-CFB, ERBB2-CNIH4, ERBB2-CTTN, ERBB2-DNAJC7, ERBB2-ENO1, ERBB2-FCGRT, ERBB2-FKBP10, ERBB2-GRB7, ERBB2-GSE1, ERBB2-IKZF3, ERBB2-KRT20, ERBB2-KRT39, ERBB2-KRTAP1-4, ERBB2-LMNTD1, ERBB2-LTBP4, ERBB2-MAD2L2, ERBB2-MED1, ERBB2-PARN, ERBB2-PGAP3, ERBB2-POLD4, ERBB2-PPP1R1B, ERBB2-PRDX4, ERBB2-PSMB3, ERBB2-SHKBP1, ERBB2-SLC39A11, ERBB2-SPTBN2, ERBB2-SRCIN1, ERBB2-TADA2A, ERBB2-TATDN1, ERBB2-XBP1, ERBB2-ZAN, ERBB4-AKAP6, ERBB4-FUS, ERBB4-IKZF2, ERBB4-STK11IP, ERC1-BRAF, ERC1-RET, ERC1-ROS1, ESRP1-RAF1, ESR1-CCDC170, ETV6-FGFR3, ETV6-NTRK2, ETV6-NTRK3, ETV6-PDGFRB, ETV6-PRDM16, EZR-ERBB4, EZR-ROS1, FAM131B-BRAF, FAT1-NTRK3, FGFR2-BICC1, FGFR2-TACC3, FGFR3-TACC3, FIP1L1-PDGFRA, FN1-ALK, FN1-ERBB4, FN1-FGFR1, FNDC3B-PIK3CA, FRY-NTRK3, GKAP1-NTRK2, GOLGA4-RAF1, GON4L-NTRK1, GOPC-ROS1, GRHL2-RSPO2, GRIPAP-NTRK1, GTF2IRD1-ALK, HACL1-RAF1, HIP1-ALK, HNRNPA2B1-NTRK3, IKZF2-ERBB4, IQSEC1-RAF1, IRF2BP2-NTRK1, KANK1-NTRK2, KCTD16-NTRK2, KCTD8-NTRK2, KHDRBS1-NTRK3, KIAA1549-BRAF, KIF5B-ALK, KIF5B-RET, KIF5B-ERBB4, KIT-ANKRD11, KIT-PDGFRA, KIT-SLC4A4, KMT2A-AFF1, KMT2A-CREBBP, KMT2A-DAB21P, KMT2A-ELL, KMT2A-EPS15, KMT2A-MLLT1, KMT2A-MLLT10, KMT2A-MLLT11, KMT2A-MLLT3, KMT2A-MLLT4, KMT2A-SEP6, KMT2A-SEP9, KTN1-ALK, KTN1-RET, LIPI-NTRK1, LMNA-ALK, LMNA-NTRK1, LM NA-RAF1, LRRC71-NTRK1, LRRFIP1-FGFR1, LRRFIP1-MET, LYN-NTRK3, MAG13-AKT3, MBNL1-RAF1, MEF2D-NTRK1, MET-MET, MIR548F1-NTRK1, MKRN1-BRAF, MPRIP-ALK, MPRIP-NTRK1, MPRIP-RAF1, MPRIP-RET, MRPL24-NTRK1, MSN-ALK, MSN-ROS1, MTSS1-ERBB2, MUC2-NTRK2, MYH9-ALK, MYO5A-NTRK3, MYO5A-ROS1, NACC2-NTRK2, NAV1-NTRK2, NBPF20-NTRK2, NCOA4-RET, NFASC-NTRK1, NOS1AP-NTRK1, NOS1AP-NTRK2, NRG2-CYSTM1, NRG2-UBE2D2, NRIP1-RSPO2, P2RY8-NTRK1, PAIP1-NTRK2, PAN3-NTRK2, PAPD7-RAF1, PDE4DIP-NTRK1, PEAR1-NTRK1, PHF20-NTRK1, PICALM-BRAF, PICALM-RET, PLEKHA6-NTRK1, PML-RARA, PPFIBP1-ALK, PPFIBP1-MET, PPFIBP1-ROS1, PPL-NTRK1, PRDX1-NTRK1, PRKAR1A-ALK, PRKAR1A-RET, PRKAR1B-ALK, PRKAR1B-BRAF, PRKAR2A-NTRK2, PRPSAP1-NTRK3, PTPRZ1-MET, QKI-NTRK2, QKI-RAF1, RAC1-AKT3, RAF1-ACTR2, RAF1-AGGF1, RAF1-DAZL, RAF1-ESRP1, RAF1-PHC3, RAF1-TMEM40, RAF1-TRAK1, RAF1-ZPR1, RALGPS2-NTRK3, RANBP2-ALK, RANBP2-FGFR1, RBPMS-NTRK3, RFWD2-NTRK1, RNF213-ALK, RNF213-NTRK1, RRBP1-ALK, RRBP1-RET, SATB1-ALK, SATB1-RET, SCAF11-PDGFRA, SCP2-NTRK1, SCYL3-NTRK1, SDC4-NRG1, SDC4-ROS1, SEC31A-ALK, SHC1-ERBB2, SIL1-NRG2, SLC34A2-MET, SLC34A2-ROS1, SLC45A3-BRAF, SLC45A3-ERG, SLC45A3-FGFR2, SLMAP-NTRK2, SND1-BRAF, SPECC1L-NTRK2, SPECC1L-NTRK3, SPTBN1-ALK, SQSTM1-ALK, SQSTM1-FGFR1, SQSTM1-NTRK1, SQSTM1-NTRK2, SQSTM1-NTRK3, SRGAP3-RAF1, SRGAP3-SRGAP3-RAF1, SSBP2-NTRK1, STRN-ALK, STRN-NTRK2, STRN-NTRK3, STRN3-BRAF, STRN3-NTRK1, STRN3-NTRK2, STRN3-NTRK3, TBC1D2-NTRK2, TBL1XR1-NRG1, TBL1XR1-PIK3CA, TBL1XR1-RET, TFG-ALK, TFG-MET, TFG-NTRK1, TFG-NTRK3, TFG-RET, TFG-ROS1, TIMP3-ALK, TIMP3-NTRK1, TKT-ERBB2, TLE4-NTRK2, TMEM106B-BRAF, TMEM106B-ROS1, TMPRSS2-ERG, TMPRSS2-ETV1, TMPRSS2-ETV4, TMPRSS2-ETV5, TNS3-NTRK2, TP53-NTRK1, TPM3-ALK, TPM3-NTRK1, TPM3-ROS1, TPM4-ALK, TPM4-NTRK3, TPR-ALK, TPR-BRAF, TPR-FGFR1, TPR-MET, TPR-NTRK1, TRAF2-NTRK2, TRAK1-RAF1, TRIM24-BRAF, TRIM24-FGFR1, TRIM24-NTRK2, TRIM24-RET, TRIM33-RET, TRIM33-NTRK1, TRIM4-BRAF, TRIM4-MET, TRIM63-NTRK1, UBE2R2-NTRK3, UFD1-NTRK2, USP13-PIK3CA, VANGL2-NTRK1, VCAN-NTRK2, VCL-ALK, VCL-NTRK2, VIM-NTRK3, VPS18-NTRK3, WHSC1L1-FGFR1, WHSC1L1-NUTM1, WIPF2-ERBB2, WNK2-NTRK2, ZBTB7B-NTRK1 or ZNF710-NTRK3.

In one preferred embodiment, the detectable NTRK gene fusion types include TFG-NTRK1, ETV6-NTRK3, QKI-NTRK2, TPM3-NTRK1, ETV6-NTRK2, TFG-NTRK3, and NACC2-NTRK2. In another preferred embodiment, the detectable NTRK gene fusion types include PDE4DIP-NTRK1, TRIM63-NTRK1, GON4L-NTRK1, and CTRC-NTRK1.

In one preferred embodiment, the detectable DNA fragment joining events include that the target DNA fragment, which includes the NTRK gene, is the downstream DNA fragment, and a partner DNA fragment, which includes the partner gene as described in the preceding paragraphs, is the upstream DNA fragment.

In one preferred embodiment, the detectable DNA fragment joining events include that the target DNA fragment, which includes the EGFR gene, is the upstream DNA fragment, and a partner DNA fragment, which includes the partner gene as described in the preceding paragraphs, is the downstream DNA fragment.

In some embodiments, the gene mutation of alternative splicing events that can be detectable includes, but is not limited to, AR (e.g. ARV7), BCL2L1, BCL2-Like 11 (BIM or BCL2L11), BCOR, BCR-ABL, BIN1, BRAF, BRCA1, BRCA2, CASP2 (CASP-2), CD19, CD44, CXCR3, Cyclin D1 (CCND1), DMP1, CDH1 (E-cadherin), EGFR (e.g. EGFRvIII), ER (e.g. ESR1 or ESR2), EZH2, FAS, FGFR2, HRAS (H-RAS), IKZF1, KLF6, KRAS, MAP3K7, MCL1, MDM4, MET, MNK2, PIK3CD, PKM, RASGRP2, RON, RPS6KB (e.g. RPS6KB1 or RPS6KB2), STAT3, TP53, TSC2, or VEGF.

In some embodiments, the first split probe is complementary to the partner DNA fragment having a sequence of the partner gene as described in the preceding paragraphs and any complementary sequence thereof. In other embodiments, the second split probe is complementary to the target DNA fragment having a sequence of the target gene as described in the preceding paragraphs and any complementary sequence thereof. In other embodiments, the third split probe is complementary to the third DNA fragment having a sequence of the partner gene or target gene as described in the preceding paragraphs and any complementary sequence thereof.

Table 1 lists the specifically designed split probes for detection of the specific DNA fragment joining. Each of these probes has been designed to be specific for the target regions of DNA fragments (ex. target gene or partner gene) and can be universally used in different DNA fragment types, allowing the modification process of probe easier and accurate determination of the DNA fragment joining events.

TABLE 1

| | Probe Sequence | SEQ ID |
|---|---|---|
| Probe Target Region | (from the 5′-end to the 3′-end) | NO |
| ABL (exon 2) | GCGGCCAGTAGCATCTGACT | 1 |
| ABL (intron 8) | TGATAACCGTGAAGAAAGAA | 2 |
| AFAP1 (exon 4) | CTCCGGAATACATCACATCA | 3 |
| ALK (exon 20) | TGTACCGCCGGAAGCACCAGGAGC | 4 |
| AR (exon 3) | GCAATTGCAAGCATCTCA | 5 |
| AR (exon 4) | AGGAAAAATTGTCCATCTTGTCGTCTTCGGAAA | 6 |
| AXL (exon 20) | TACAGAGTGTGGACTCTGGTGCCTCCAGA | 7 |
| BAG4 (exon 2) | CATATCCTGTAAGACCAGAA | 8 |
| BCR (exon 14) | GCCACTGGATTTAAGCAGAGTTCAA | 9 |
| BICC1 (exon 2) | TGCTGCTGAAGGGAAAGGCAGAAGT | 10 |
| CCDC6 (exon 1) | GAACCGCGACCTGCGCAAAGCCAGCGTGA | 11 |
| EGFR (exon 1) | GTCGGGCTCTGGAGGAAAAGAAAG | 12 |
| EGFR (exon 8) | GTAATTATGTGGTGACAGAT | 13 |
| EML4 (exon13) | CACCTGGGAAAGGACCTAA | 14 |
| FGFR1 (exon 6) | ACTCTGTGGTGCCCTCTGACAAGGGCAAC | 15 |
| FGFR2 (exon 10) | TTACAGCTTCCCCAGACTACCTGG | 16 |
| FGFR2 (exon 17) | ATTCTCACTCTCACAACC | 17 |
| FGFR2 (exon 8) | ACCAGTCTGCCTGGCTCA | 18 |
| FGFR3 (exon 17) | TGTCCTTACCGTGACGTCCACCGA | 19 |
| MBIP (exon 4) | AAGCTGAAATCAATGAAAACAACGTCAGGGA | 20 |
| MET (exon 13) | TGTGGCTGAAAAAGAGAAAGCAAATTAAAG | 21 |
| MET (exon 15) | ATCAGTTTCCTAATTCATCT | 22 |
| NTRK1 (exon 10) | ACAGCACATCTGGAGACCCG | 23 |
| PDGFRA (exon 2) | TTTCCCAGAGCTATGGGGACTT | 24 |
| PPL (exon 22) | AATAAACCTGGTGGCTGGAG | 25 |
| RET (exon 12) | AAGTGGGAATTCCCTCGGAAGA | 26 |
| ROS1 (exon 32) | TCCCAAATTACTAGAAGGGA | 27 |
| SCAF11 (exon 1) | CCTCGACCTCGGTCTGA | 28 |
| SLC34A2 (exon 4) | TCGTGTGCTCCCTGGATATTCTTA | 29 |
| TACC3 (exon 8) | AGTCGGCCTTGAGGAAGCA | 30 |

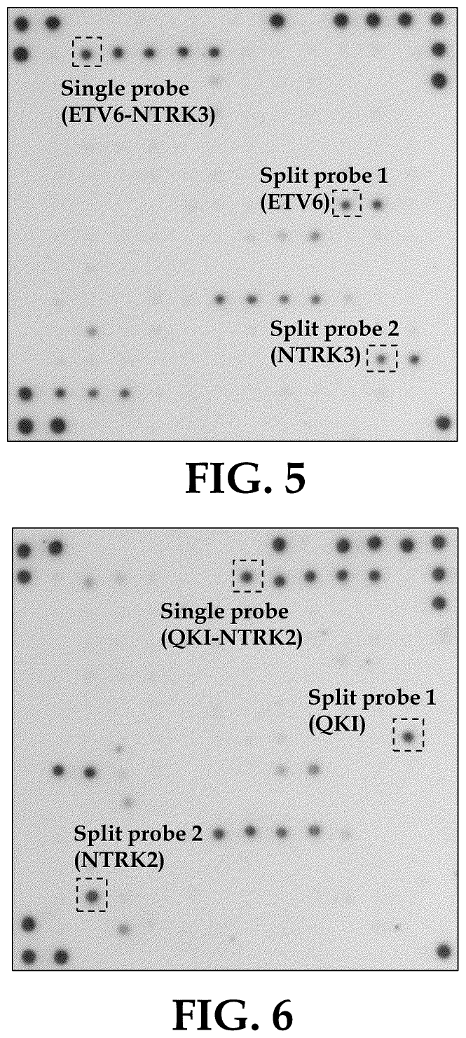

In some embodiments, for detection of the NTRK gene fusion, the single probes having any of the sequences of 5′-GGGAGAATAGCAGGTCCCGT-3′ (SEQ ID NO: 31) or 5′-TGGTGTATTAGGCCCAGCCT-3′ (SEQ ID NO: 34) are used to compare detection sensitivity and specificity with the split probes having any of the sequences of SEQ ID NOs: 32, 33, 35, 36 (refer to TABLE 2, FIG. 5, and FIG. 6).

TABLE 2

| | Probe Sequence | |
|---|---|---|
| Probe Target | (from the 5′-end to | |
| Region | the 3′-end) | SEQ ID NO |
| ETV6 (exon 5) | CGCCATGCCCATTGGGAGAA | 32 |
| NTRK3 (exon 14) | GTCCCGTGGCTGTCATCAGT | 33 |
| QKI (exon 6) | TATCCTATTGAACCTAGTGG | 35 |
| NTRK2 (exon 16) | GCCCAGCCTCCGTTATCAGC | 36 |

In some embodiments, one DNA fragment joining event is detected after one target amplified nucleic acid is amplified and probed. In other embodiments, multiple NTRK fusion types can be detected simultaneously after two or more target nucleic acids with difference sequences are amplified in one reaction (called a multiplex amplification reaction) and/or probed in one reaction (called a multiplex hybridization reaction). In some embodiments, at least two sets of the split probes are selected from the group consisting of SEQ ID NOs: 32, 33, 35, 36, and any complementary sequence thereof. The probes may be provided as a single pooled reagent or as separate reagents.

Typically, the probe and the amplified product are mixed at a specific temperature to facilitate probe hybridization. The optimal thermal mixing condition for probe hybridization varies depending on probe sequences. Thus, for a multiplex reaction where at least two probes are applied, it is difficult to select a suitable hybridization condition for all the probes. However, by using the probes listed in TABLE 2, a multiplex reaction may be performed at a fixed temperature with agitation at a fixed speed, because these probes are designed to be capable of hybridizing to the respective target at similar hybridization conditions. In some embodiments, the temperature for hybridization is between 35-50° C., 40-50° C., 40-45° C., or 45-50° C. In some embodiments, the hybridization is performed by using a thermomixer at a rotation speed between 700-1000 rpm, 750-1000 rpm, 800-1000 rpm, 900-1000 rpm, 700-750 rpm, 700-800 rpm, 750-800 rpm, or 800-900 rpm.

Detection of the probe-bound product may be accomplished through detecting the gene-specific primers, the universal primers, or the split probe in said product. Thus, the primers or probes are usually modified to be detectable. They may be modified to have fluorescence or chemiluminescence activity or become chromogenic or colorimetric by being connected directly or indirectly to a detectable molecule. In some embodiments, one or both primers in the primer pair are connected to biotin or other compounds capable of binding to a streptavidin-conjugated detectable molecule with signals. The detectable signals may be a dye, a chemiluminescent dyes, a fluorescent molecule such as phycoerythrin (PE) or cyanines, a radioisotopes, a spin labels, a haptens, a quantum dots, a beads, an aminohexyls, a pyrenes, or an enzyme for a chromogenic reaction such as alkaline phosphatase (AP) or horseradish peroxidase (HRP). The enzyme used in a chromogenic reaction catalyzes the production of colored compounds in the presence of a chromogenic substrate. In some embodiments, the split probes for detecting particular NTRK fusion types are separately connected to their unique identifier such that multiple NTRK fusion types can be detected simultaneously and distinguished from one another. The unique identifier may be an oligonucleotide with a unique sequence, a microbead, or a nanoparticle that includes a unique barcode on the surface. The barcode may be a geometric pattern that can be read by an optical scanner with a brightfield imaging system. In some embodiments, the microbead or nanoparticle is a magnetic particle. In some embodiments, the microbead or nanoparticle is made of synthetic polymers.

The unique identifier may be connected to the probe directly or through a linker. In some embodiments, the unique identifier is connected to the probe by direct chemical coupling, and a covalent bond is formed therebetween. In some embodiments, the unique identifier is connected to the probe through a polymer linker. In some embodiments, the unique identifier is connected to the probe by hybridization between complementary nucleotide sequences.

The disclosed method can be performed on several technology platforms capable of running multiplex reactions, such as a microarray plate, a gene chip, microbeads, nanoparticles, a membrane, or a microfluidic device. In some embodiments, the probes are immobilized on a microarray plate, a gene chip, or a membrane at different positions, for example, in the form of an array of spots, each containing multiple copies of one type of probe. In other embodiments, the probes are coupled with microbeads (such as micro magnetic beads). In still other embodiments, the probes are coated on a substrate plate of a microfluidic device, in which different probes are placed in different regions of the substrate plate.

When the probes are immobilized on a DNA microarray plate, the microarray plate may further include a set of control spots, each containing multiple copies of a control probe. The control probe binds the DNA of housekeeping genes such as beta-actin, glyceraldehyde 3-phosphate dehydrogenase (GAPDH), and beta 2-microglobulin. Thus, the control spots can be used as an internal control to validate assay performance. In addition, the microarray plate may further include a set of anchor spots, each containing multiple copies of an anchor probe. The anchor probe is designed to be detected irrespective of the amplified products. Thus, the anchor spots can be used as a position indicator for nearby spots on the microarray plate.

In the present disclosure, a method is also provided for treating a subject. Said method includes steps of
(a) determining whether a subject is at risk of cancer or genotype, including detecting a DNA fragment joining event by the method as described in the preceding paragraphs and/or distinguishing an alternative splicing event by the method as described in the preceding paragraphs of the sample from the subject; and
(b) administering
(i) a therapeutically effective amount of a siRNA targeting the DNA fragment joining event and/or the alternative splicing event;
(ii) a therapeutically effective amount of an inhibitor of a fusion protein encoded by the DNA fragment joining event and/or the alternative splicing event;
(iii) a therapeutically effective amount of an agent that inhibits a fusion protein encoded by the DNA fragment joining event and/or the alternative splicing event;
(iv) a therapeutically effective amount of an anticancer agent selected from the group consisting of cytokines, apoptosis-inducing agents, anti-angiogenic agents, chemotherapeutic agents, radio-therapeutic agents, and anticancer immunotoxins; or
(v) providing a targeted genome editing procedure within cells of the subject.

In some embodiments, the DNA fragment joining event and/or the alternative splicing event presents with a sequence of the partner gene as described in the preceding paragraphs. In some embodiments, the DNA fragment joining event and/or the alternative splicing event presents with a sequence of the target gene as described in the preceding paragraphs.

In some embodiments, the alternative splicing event is selected from the group consisting of constitutive splicing, exon skipping, intron retention, mutually exclusive exons, and alternative 5′ or 3′ splice site.

In some embodiments, the cancer is selected from the group consisting of carcinoma, sarcoma, lymphoma, leukemia, or myeloma.

In some embodiments, the cancer is selected from the group consisting of brain cancer, breast cancer, colon cancer, endocrine gland cancer, esophageal cancer, female reproductive organ cancer, head and neck cancer, hepatobiliary system cancer, kidney cancer, lung cancer, mesenchymal cell neoplasm, prostate cancer, skin cancer, stomach cancer, tumor of the exocrine pancreas, and urinary system cancer.

In some embodiments, when NTRK gene fusions (DNA fragment joining) are detected in a sample from a cancer patient (subject), the patient is expected to respond to a TRK inhibitor, particularly an NTRK inhibitor such as larotrectinib, entrectinib, LOXO-195 or TPX-0005.

In some embodiments, the methods as disclosed in the preceding paragraphs can be used in a prospective analysis of the course or treatment of RNA splicing-related diseases or cancer diseases (See, e.g., Scotti, M., Swanson, M. RNA mis-splicing in disease. Nat Rev Genet 17, 19-32 (2016); Kim, H. K., Pham, M. H. C., Ko, K. S. et al. Alternative splicing isoforms in health and disease. Pflugers Arch—Eur J Physiol 470, 995-1016 (2018); Wang, E., & Aifantis, I. RNA Splicing and Cancer. Trends in Cancer 6, 631-644 (2020), the contents of which are incorporated herein by reference).

In the present disclosure, a kit for detecting a sample with a DNA fragment joining event and/or an alternative splicing event is also provided. The Kit includes:
(a) a set of oligonucleotides;
(b) a split probe having:
(i) a first split probe that is complementary to the 3′ end of a partner DNA fragment, a second split probe that is complementary to the 5′ end of a target DNA fragment, and/or a third the split probe that is complementary to an another DNA fragment, wherein a gap of the split probe targeting sites on a target amplified nucleic acid is within a distance of 0-80 bp from one another or
(ii) a first split probe that is complementary to the 5′ end of a partner DNA fragment, a second split probe that is complementary to the 3′ end of a target DNA fragment, and/or a third the split probe that is complementary to a third DNA fragment, wherein a gap of the split probe targeting sites on a target nucleic acid is within a distance of 0-80 bp from one another; and
(c) a probe hybridization assay for detection of the split probe hybridization signals, which includes dyes, chemiluminescent dyes, fluorescent molecules, radioisotopes, spin labels, enzymes, haptens, quantum dots, beads, aminohexyls, and pyrenes.

In some embodiments, the kit includes the set of oligonucleotides which is a gene-specific primer or a gene-specific probe. In some embodiments, the kit includes at least two pairs of the gene-specific primers as described in the preceding paragraphs. In some embodiments, the gene-specific primer is designed to obtain the target nucleic acid from which the partner DNA fragment as an upstream DNA fragment. In some embodiments, the gene-specific primer is designed to obtain the target nucleic acid from which the partner DNA fragment as a downstream DNA fragment.

In some embodiments, the kit further includes a universal primer as described in the preceding paragraphs.

In some embodiments, at least one of the gene-specific primers targets a DNA fragment joining boundary.

In some embodiments, the gene-specific primer targets within a distance of 0-80 bp from a DNA fragment joining boundary.

In some embodiments, the first split probe and the second split probe target within a distance of 0-40 bp from a DNA fragment joining boundary.

In some embodiments, the first split probe is complementary to the partner DNA fragment having a sequence of the partner gene as described in the preceding paragraphs and any complementary sequence thereof. In other embodiments, the second split probe is complementary to the target DNA fragment having a sequence of the target gene as described in the preceding paragraphs and any complementary sequence thereof. In other embodiments, the third split probe is complementary to the third DNA fragment having a sequence of the partner gene or target gene as described in the preceding paragraphs and any complementary sequence thereof.

In some embodiments, the first split probe is complementary to the partner DNA fragment, which includes a sequence of the partner gene as described in the preceding paragraphs and any complementary sequence thereof. In some embodiments, the second split probe is complementary to the target DNA fragment, which includes a sequence of the target gene as described in the preceding paragraphs and any complementary sequence thereof. In some embodiments, the third split probe is complementary to the third DNA fragment which comprises a sequence of the partner gene or the target gene as described in the preceding paragraphs and any complementary sequence thereof.

In some embodiments, the split probes are selected from the group consisting of SEQ ID NOs: 32, 33, 35, 36, and any complementary sequence thereof.

In some embodiments, one or both primers in the primer pair are connected to biotin or other compounds capable of binding to a streptavidin-conjugated detectable molecule with signals.

In some embodiments, a length of the split probe is 10-60 bp.

In some embodiments, the length of the target amplified nucleic acid is no longer than 200 bp.

In some embodiments, a probe hybridization assay is designed for use in a variety of detectable signals such as a dye, a chemiluminescent dyes, a fluorescent molecule such as phycoerythrin (PE) or cyanines, a radioisotopes, a spin labels, a haptens, a quantum dots, a beads, an aminohexyls, a pyrenes, or an enzyme for a chromogenic reaction such as alkaline phosphatase (AP) or horseradish peroxidase (HRP).

In some embodiments, the split probes for detecting particular NTRK fusion types are designed connected to their unique identifier. The unique identifier may be an oligonucleotide with a unique sequence, a microbead, or a nanoparticle that includes a unique barcode on the surface. The barcode may be a geometric pattern that can be read by an optical scanner with a brightfield imaging system. In some embodiments, the microbead or nanoparticle is a magnetic particle. In some embodiments, the microbead or nanoparticle is made of synthetic polymers.

In some preferred embodiments, the kit includes a universal primer. When the pairs of the NTRK fusion-specific primer are used in combination with the universal primer, the NTRK fusion-specific forward primer in each pair of the NTRK fusion-specific primer further encompasses the nucleotide sequence of the universal forward primer in the pair of the universal primers, and the NTRK fusion-specific reverse primer in each pair of the NTRK fusion-specific primer further encompasses the nucleotide sequence of the universal reverse primer in the pair of the universal primer.

In some embodiments, the kit further includes a reverse transcriptase for reverse transcription of the RNA isolated from the sample, and also includes a DNA polymerase for amplification of the cDNA generated by the reverse transcription.

In some embodiments, the kit further includes an internal control. The internal control may be a positive control sample where an NTRK gene fusion is present or may be a negative control sample having no NTRK gene fusion. In some embodiments, the internal control is an FFPE tissue section, peripheral blood mononuclear cells (PBMCs), blood, plasma, other cells or body fluids, nucleic acids, or oligonucleotides.

The kit as described in the preceding paragraphs takes advantage of the detecting accuracy in finding the joining of DNA fragment and the alternative splicing event, resulting in the reliable analysis of the clinical genotype.

The present disclosure is further illustrated by the following Examples, which are provided for demonstration rather than limitation.

### EXAMPLE 1

### Detection of MET Gene Mutation by a One-Step PCR Target-Probe Hybridization Assay

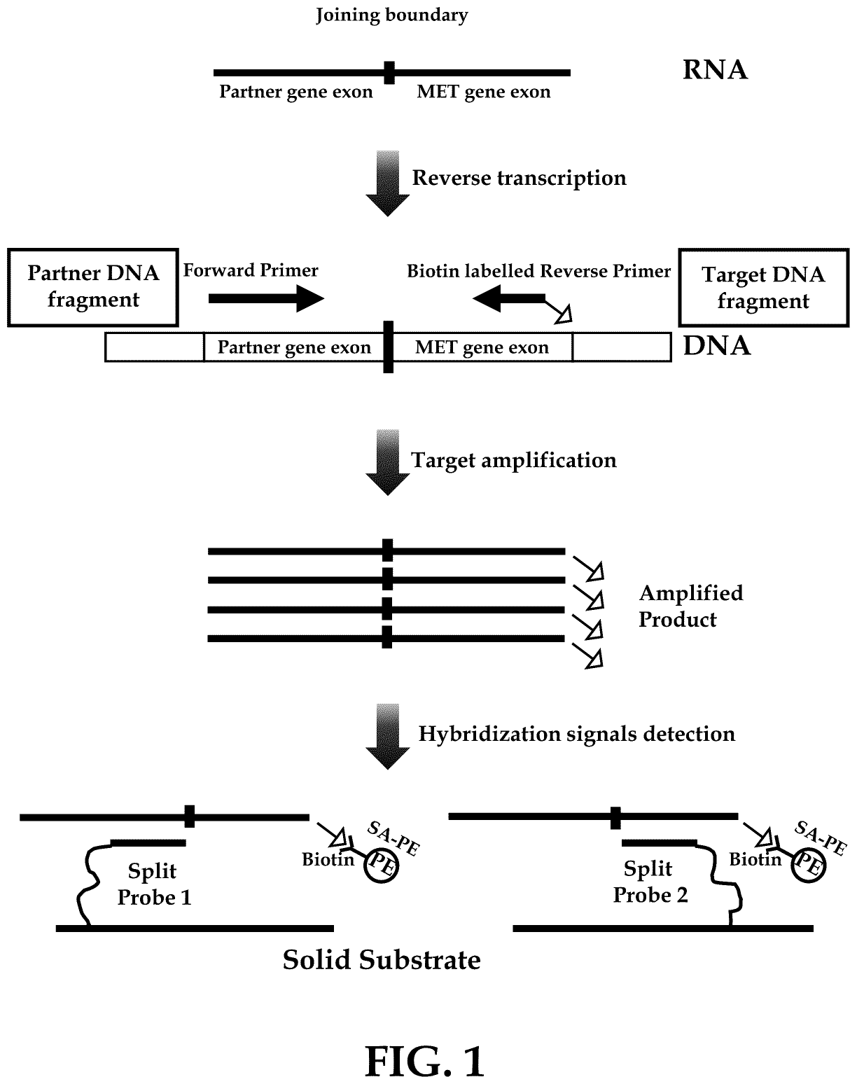

One-step PCR target-probe hybridization assay can simultaneously detect possible MET alternative splicing types in a single reaction. FIG. 1 shows the overall process of this assay, which includes the steps of obtaining RNA from a sample, reverse transcription of the RNA to obtain cDNA, PCR amplification of MET alternative splicing regions of the cDNA (i.e., the target cDNA), using multiple MET mutation-specific primer pairs to obtain an amplified product of the target cDNA, PCR amplification of the first amplified product, using a universal primer pair to obtain a second amplified product of the target cDNA, probe hybridization with the amplified target cDNA by using the split probes, and detection of the probe-bound product. Below is an example of this assay.

### Preparation of the Split Probes

Before the assay, a probe targeting MET exon 14 skipping mutation is designed based on the nucleotide sequence of the fusion region in the RNA transcript of the MET gene (Table 3). The sequence in Table 3 is from the 5′ partner (exon 13 of the MET gene) and the 3′ target (exon 15 of the MET gene), respectively. The split probe (e.g. as shown in Table 1) is designed to bind to the sequence listed in Table 3. The split probes are immobilized on a microarray plate, in the form of an array of spots, each containing multiple copies of one type of probe.

TABLE 3

| | | | Sequence of the fragment joining region | SEQ ID |
|---|---|---|---|---|
| Mutation Type | Partner and Target | | (from the 5′ end to the 3′ end) | NO |
| MET-MET | MET | MET | TGGAAGCAAGCAATTTCTTCAACCGTCCTTGG | 37 |
| alternative | exon 13 | exon 15 | AAAAGTAATAGTTCAACCAGATCAGAATTTC | |
| splicing | | | ACAGGATTGATTGCTGGTGTTGTCTCAATATC | |
| | | | AACAGCACTGTTATTACTACTTGGGTTTTTCC | |
| | | | TGTGGCTGAAAAAGAGAAAGCAAATTAAAG | |
| | | | ATCAGTTTCCTAATTCATCTCAGAACGGTTCA | |
| | | | TGCCGACAAGTGCAGTATCCTCTGACAGACA | |
| | | | TGTCCCCCATCCTAACTAGTGGGGACTCTGAT | |
| | | | ATATCCAGTCCATTACTGCAAAATACTGTCCA | |
| | | | CATTGACCTCAGTGCTCTAAATCCAGAGCTG | |
| | | | GTCCAGGC | |

### PCR Enrichment by Using MET Mutation-Specific Primer Pair

To substitute clinical samples harboring MET alternative splicing, oligonucleotides are synthesized by IDT to be used as a positive control template. The MET alternative splicing oligo is amplified by PCR with a MET mutation-specific primer pair shown in Table 4. This primer pair, capable of binding to the 5′-end and the 3′-end of the MET alternative splicing oligo, is synthesized by IDT. The reverse primer in the primer pair is modified at the 5′-end with biotin for subsequent interaction with a streptavidin-phycoerythrin (SA-PE) conjugate (Thermo Fisher Scientific). The PCR is performed on Veriti™ 96-Well Thermal Cycler (Thermo Fisher Scientific) for 30 thermal cycles using Platinum Taq DNA polymerase High Fidelity (Thermo Fisher Scientific) according to the manufacturer's instructions.

TABLE 4

| | Sequence of the MET mutation- | | SEQ |
|---|---|---|---|
| Mutation | specific primer pair (from the 5′ | | ID |
| Type | end to the 3′ end) | | NO |
| MET-MET | Forward | AAGAGAAAGCAAATTAAAGATCAGTT | 38 |
| alternative | Reverse | CTGTCAGAGGATACTGCAC | 39 |
| splicing | | | |

### Probe Hybridization and Signal Detection

The amplified product of the MET alternative splicing oligo is transferred to pre-blocked wells for hybridization, each of which is printed with an array of split probe spots, including the spots of MET alternative splicing-specific probes (e.g. as shown in Table 1), the spots of control probes, and the spots of an anchor probe. After the hybridization, a fluorescent SA-PE conjugate is subsequently added to the wells to bind the biotin of the amplified product, so that color products form at the position where a probe-target hybrid is present. By photographing the wells with a specific camera and identifying the position of color spots in the wells, the particular hybridization indicating the presence of a particular MET alternative splicing can be determined. The position of color spots can be analyzed by a computer.

### EXAMPLE 2

### Detection of NTRK Gene Mutation by a Two-Step PCR Target-Probe Hybridization Assay

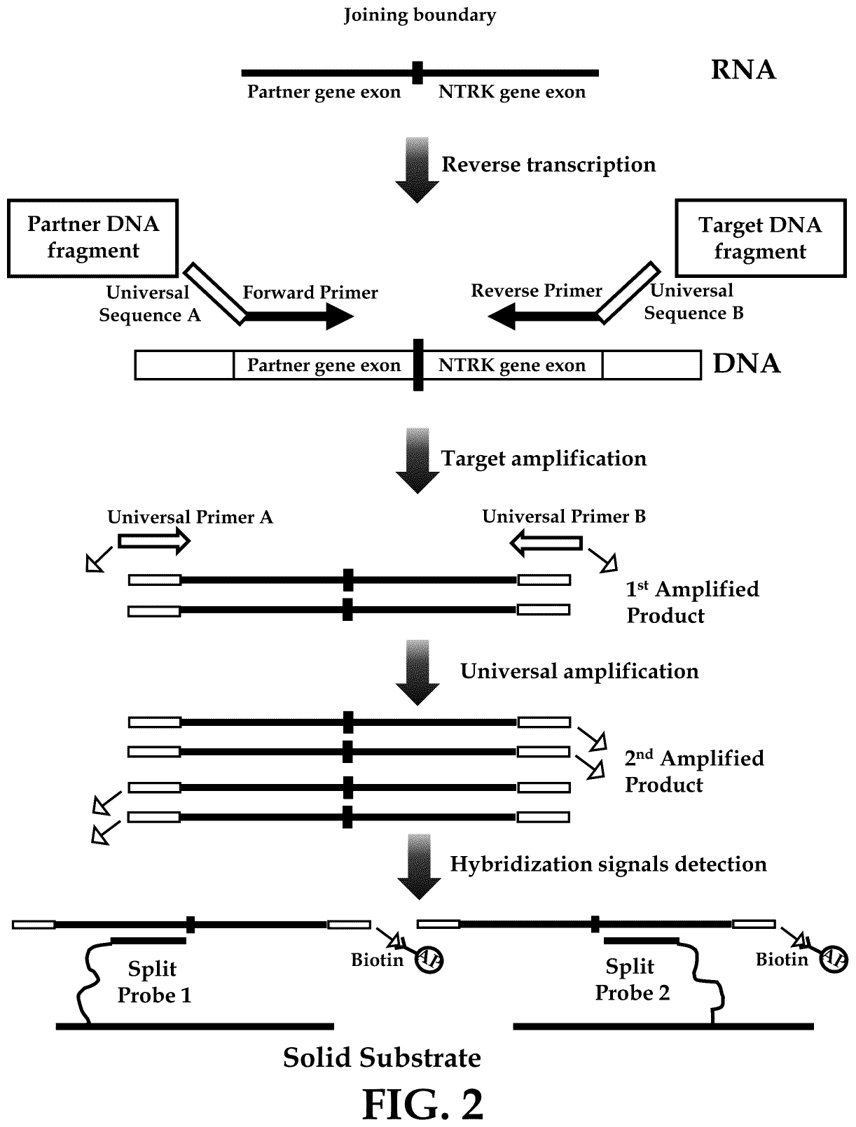

Two-step PCR target-probe hybridization assay is another method designed for simultaneous detection of multiple possible NTRK fusions in a single reaction. FIG. 2 shows the overall process of this assay, which includes the steps of obtaining RNA from a sample, reverse transcription of the RNA to obtain cDNA, PCR amplification of NTRK fusion regions of the cDNA (i.e., the target cDNA) using multiple NTRK fusion-specific primer pairs to obtain a first amplified product of the target cDNA, PCR amplification of the first amplified product using a universal primer pair to obtain a second amplified product of the target cDNA, probe hybridization with the amplified target cDNA and detection of the probe-bound product. Below is an example of this assay.

### RNA Extraction and Reverse Transcription

Both DNA and RNA are extracted from an FFPE tissue specimen from a cancerous patient by using RecoverAll total nucleic acid isolation kit (Cat No: AM1975, Ambient Technologies) according to the manufacturer's instructions. Reverse transcription of 100 ng total RNA is carried out at 42° C. for 30 to 60 minutes by using SuperScript cDNA synthesis kit (Cat No: 11754050, Invitrogen) and random hexanucleotide primers. This step yielded a cDNA product in 10 μL.

### PCR Enrichment by Using NTRK Fusion-Specific Primer Pairs

Each primer in the NTRK fusion-specific primer pair used in this assay is designed to have two segments. One segment, called a fusion-specific segment, is used to bind the 5′-end or the 3′-end of the fusion sequence of one particular NTRK fusion. The other segment, called a universal segment, encompasses the nucleotide sequence of the universal primer to be used in the second round of PCR. The universal segment is always upstream, or at the 5′ position, relative to the fusion-specific segment (FIG. 2). The universal primer may be any of the primers listed in Table 5, where each universal primer can be used as either the universal forward primer or the universal reverse primer. Table 6 shows the fusion-specific segments of some fusion-specific primer pairs used in this assay.

TABLE 5

| Universal | Primer sequence | SEQ ID |
|---|---|---|
| primer No. | (from the 5′ end to the 3′ end) | NO |
| U01 | GTTTTCCCAGTCACGACGT | 40 |
| U02 | GCAAATGGCATTCTGACATCC | 41 |
| U03 | GCGGATAACAATTTCACACAGG | 42 |
| U04 | CGTCCATGCCGAGAGTG | 43 |
| U05 | CTTTATGTTTTTGGCGTCTTCCA | 44 |
| U06 | GACTGGTTCCAATTGACAAGC | 45 |
| U07 | GCGTGAATGTAAGCGTGAC | 46 |
| U08 | TGTAAAACGACGGCCAGT | 47 |
| U09 | AAGGGTCTTGCGAAGGATAG | 48 |
| U10 | GGGTTATGCTAGTTATTGCTCAG | 49 |

TABLE 6

| | Sequence of the NTRK fusion- | | SEQ |
|---|---|---|---|
| Mutation | specific primer pairs (from | | ID |
| type | the 5′ end to the 3′ end) | | NO |
| ETV6-NTRK3 | Forward | CCACATCATGGTCTCTGTCT | 50 |
| fusion | Reverse | TGGTTGATGTGGTGCAG | 51 |
| TFG-NTRK1 | Forward | ACAGCAGCCACCATATACA | 52 |
| fusion | Reverse | AGACCCCAAAAGGTGTT | 53 |
| TFG-NTRK1 | Forward | ATCCTTTAAAAAACCAAGATGA | 54 |
| fusion | | AATCAATA | |
| | Reverse | GAGAAGGGGATGCACCA | 55 |
| TPM3-NTRK1 | Forward | GACCCGTGCTGAGTTTG | 56 |
| fusion | Reverse | AAATGCAGGGACATGGC | 57 |
| ETV6-NTRK2 | Forward | TTCCACCCTGGAAACTCTATAC | 58 |
| fusion | Reverse | CATTGGAGATGTGATGGAGTG | 59 |
| TFG-NTRK3 | Forward | ACAGCAGCCACCATATACA | 60 |
| fusion | Reverse | CTCGATGCAGTGCTCCA | 61 |

For fusion-specific PCR, 7 μL water is added to 10 μL of the cDNA product, and the resulting mixture (17 μL) is subsequently divided into four equal pools of 4 μL mixture each, leaving 1 μL as dead volume. The number of pools is decided based on primer performance. More specifically, the primer efficiency of each fusion-specific primer pair is determined first, and the fusion-specific primer pairs with similar efficiencies are mixed to form a single primer pool, resulting in a total of four primer pools (denoted as P1, P2, P3, and P4). Each primer pool, containing 23 to 48 fusion-specific primers (Table 7), is added into one pool of cDNA (4 μL). The cDNA in each pool is then amplified on Veriti™ 96-Well Thermal Cycler (Thermo Fisher Scientific) for 25 thermal cycles using multiplex PCR kit (Cat No: 206143, Qiagen) according to the manufacturer's instructions, yielding a first amplified product in 10 μL. In other words, four multiplex PCR reactions are performed to yield four pools of the first amplified products.

TABLE 7

| | Number of | Number of NTRK | Number of |
|---|---|---|---|
| Pool No. | primers | fusion genes | housekeeping gene |
| P1 | 48 | 39 | 1 |
| P2 | 35 | 25 | 1 |
| P3 | 43 | 28 | 1 |
| P4 | 23 | 16 | 0 |

### PCR Enrichment by Using a Universal Primer Pair

Since each fusion-specific primer included the nucleotide sequence of a universal primer at the 5′ end, the first amplified products can be further amplified by PCR using a universal primer pair, including a universal forward primer with the sequence selected from SEQ ID NOs: 40-49 and a universal reverse primer with the sequence selected from SEQ ID NOs: 40-49. The universal reverse primer is biotinylated. For the second round of PCR, each of the four pools of the first amplified products is diluted 100 folds in the final reaction mix and amplified on Veriti™ 96-Well Thermal Cycler (Thermo Fisher Scientific) for 25 thermal cycles using Platinum SuperFi II PCR Master Mix (Cat No: 12368010, Invitrogen) according to the manufacturer's instructions, yielding a second amplified product in 10 μL. In other words, four PCR reactions are performed to yield four pools of the second amplified products.

### Probe Hybridization and Signal Detection

The four pools of the second amplified products (a total of 40 μL) are combined, and 18 μL of the resulting pool is mixed with 3 μL water to yield a mixture. The mixture is placed in a 96-well PCR plate (Cat No: P46-4TI-1000/C, 4titude). The second amplified products are denatured at 96° C. for 5 minutes and transferred to pre-blocked wells, each of which is previously printed with an array of probe spots, including the spots of the split probes, 9 spots of control probes, and 10 spots of an anchor probe. The split probe is selected from the sequences of SEQ ID NOs: 32, 33, 35, 36 (Table 2). FIG. 5 and FIG. 6 show the distribution of different probes in one well. Target-probe hybridization is performed at 50° C. for 15 minutes with vibration. After the hybridization, the wells are cooled and washed twice. A buffer containing a streptavidin-alkaline phosphatase conjugate is subsequently added to the wells to allow biotin-streptavidin interaction, and a substrate for the alkaline phosphatase is then added so that color product formed at the position where a probe-target hybrid is present. By photographing the wells with a camera and identifying the position of color spots in the wells, the particular hybridization indicating the presence of a particular NTRK fusion is determined. The position of color spots can be analyzed by a computer.

### EXAMPLE 3

### Detection of EGFRvIII Mutation by a Two-Step PCR Target-Probe Hybridization Assay

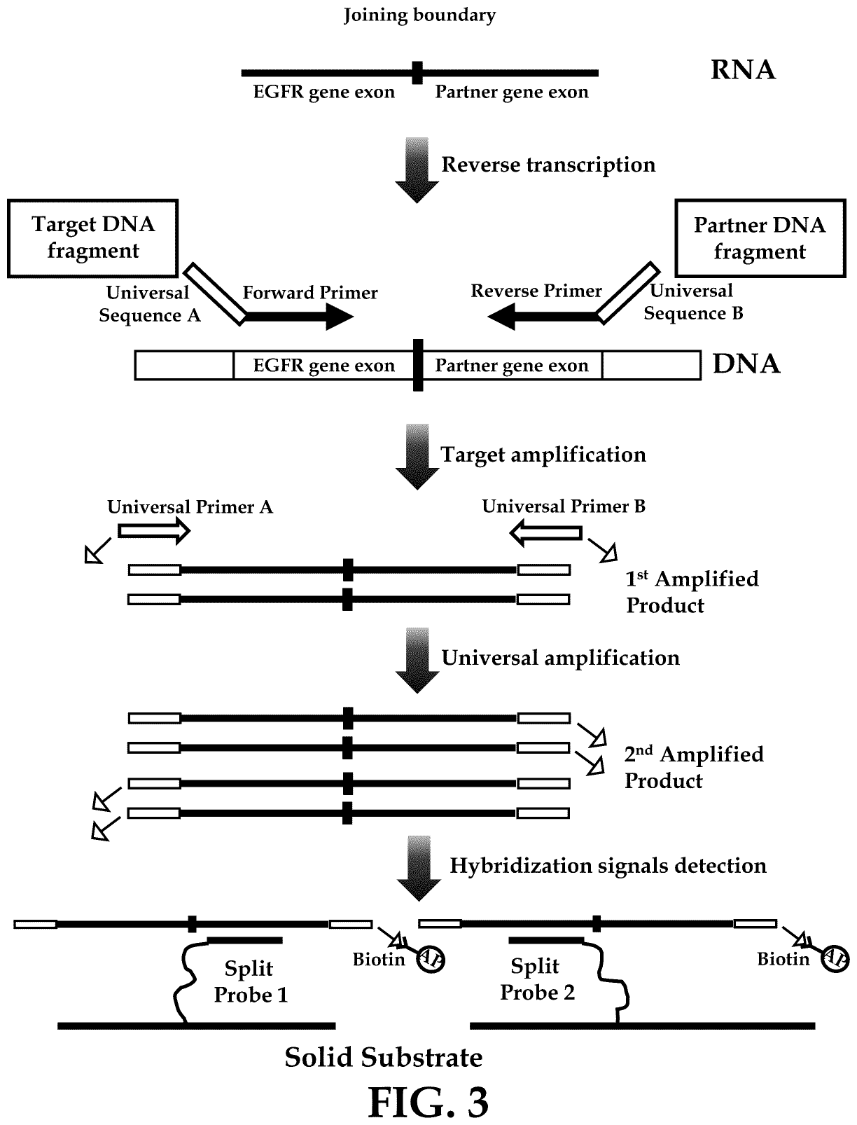

A two-step PCR target-probe hybridization assay is used here to detect EGFRvIII mutations. FIG. 3 shows the overall process of this assay, which includes the steps of obtaining RNA from a sample, reverse transcription of the RNA to obtain cDNA, PCR amplification of the EGFRvIII mutation region of the cDNA (i.e., the target cDNA) using an EGFRvIII mutation-specific primer pair to obtain a first amplified product of the target cDNA, PCR amplification of the first amplified product using a universal primer pair to obtain a second amplified product of the target cDNA, probe hybridization with the amplified target cDNA and detection of the probe-bound product. Below is an example of this assay.

### RNA Extraction and Reverse Transcription

Both DNA and RNA are extracted from an FFPE tissue specimen from a cancerous patient by using RecoverAll total nucleic acid isolation kit (Cat No: AM1975, Ambient Technologies) according to the manufacturer's instructions. Reverse transcription of 100 ng of total RNA is carried out at 42° C. for 30 to 60 minutes by using SuperScript cDNA synthesis kit (Cat No: 11754050, Invitrogen) and random hexanucleotide primers, and 10 μL of cDNA product is obtained.

### PCR Enrichment by Using an EGFRvIII Mutation-Specific Primer Pair

Each primer in the EGFRvIII mutation-specific primer pair used in this assay is designed to have two segments. One segment, called an alternative splicing specific segment, is used to bind the 5′-end or the 3′-end of the EGFRvIII mutation sequence. The alternative splicing specific segment may have the sequence of SEQ ID NO:62 or 63 (Table 8). The other segment, called a universal segment, encompasses the nucleotide sequence of the universal primer to be used in the second round of PCR. The universal segment is always upstream, or at the 5′ position, relative to the alternative splicing specific segment. The universal primer may be any of the primers listed in Table 5, where each universal primer can be used as either the universal forward primer or the universal reverse primer.

TABLE 8

| | Sequence of the EGFRvIII | | SEQ |
|---|---|---|---|
| Mutation | mutation-specific primer pair | | ID |
| type | (from the 5′ end to the 3′ end) | | NO |
| EGFR-EGFR | Forward | GGGCTCTGGAGGAAAAGAA | 62 |
| alternative | Reverse | TCCATCTCATAGCTGTCGG | 63 |
| splicing | | | |

For alternative splicing specific PCR, 10 uL of the cDNA product is amplified on Veriti™ 96-Well Thermal Cycler (Thermo Fisher Scientific) for 15-30 thermal cycles using multiplex PCR kit (Cat No: 206143, Qiagen) according to the manufacturer's instructions, yielding a first amplified product in 10 μL.

### PCR Enrichment by Using a Universal Primer Pair

Since each mutation-specific primer included the nucleotide sequence of a universal primer at the 5′ end, the first amplified product can be further amplified by PCR using a universal primer pair, including a universal forward primer with the sequence selected from SEQ ID NOs: 40-49 and a universal reverse primer with the sequence selected from SEQ ID NOs: 40-49. The universal reverse primer is biotinylated. For the second round of PCR, the first amplified product is diluted 100 folds in the final reaction mix and amplified on Veriti™ 96-Well Thermal Cycler (Thermo Fisher Scientific) for 15-30 thermal cycles by using Platinum SuperFi II PCR Master Mix (Cat No: 12368010, Invitrogen) according to the manufacturer's instructions, yielding a second amplified product in 10 μL.

### Probe Hybridization and Signal Detection

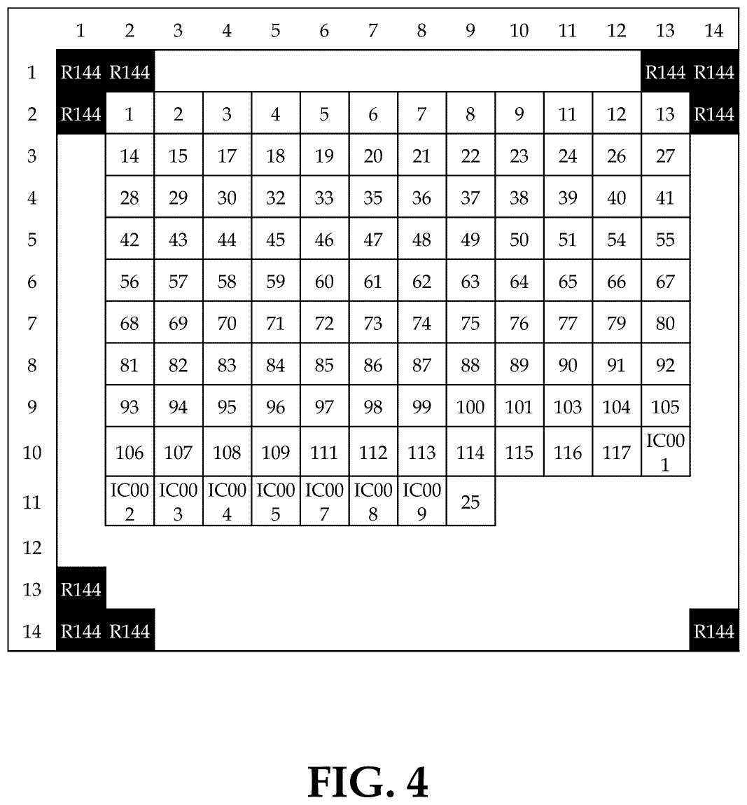

The second amplified product is placed in a 96-well PCR plate (Cat No: P46-4TI-1000/C, 4titude). The second amplified products are denatured at 96° C. for 5 minutes and transferred to pre-blocked wells, each of which is previously printed with an array of probe spots, including 117 spots of the split probes, 9 spots of control probes, and 10 spots of an anchor probe (FIG. 4). The split probe (e.g. as shown in Table 1) is designed to bind to the sequence listed in Table 9. Target-probe hybridization is performed at about 50° C. for 15 minutes with vibration. After the hybridization, the wells are cooled and washed twice. A buffer containing a streptavidin-alkaline phosphatase conjugate is subsequently added to the wells to allow biotin-streptavidin interaction, and a substrate for the alkaline phosphatase is then added so that color products form at the position where a probe-target hybrid is present. By photographing the wells with a camera and identifying the position of color spots in the wells, the particular hybridization indicating the presence of EGFRvIII can be determined. The position of color spots can be analyzed by a computer.

TABLE 9

| | | | Sequence of the fragment joining region | SEQ ID |
|---|---|---|---|---|
| Mutation type | Partner and Target | | (from the 5′ end to the 3′ end) | NO |
| EGFR-EGFR | EGFR | EGFR | CCACCTCGTCGGCGTCCGCCCGAGTCCCCGC | 64 |
| alternative splicing | exon 1 | exon 8 | CTCGCCGCCAACGCCACAACCACCGCGCACG | |
| | | | GCCCCCTGACTCCGTCCAGTATTGATCGGGA | |
| | | | GAGCCGGAGCGAGCTCTTCGGGGAGCAGCG | |
| | | | ATGCGACCCTCCGGGACGGCCGGGGCAGCG | |
| | | | CTCCTGGCGCTGCTGGCTGCGCTCTGCCCGG | |
| | | | CGAGTCGGGCTCTGGAGGAAAAGAAAGGTA | |
| | | | ATTATGTGGTGACAGATCACGGCTCGTGCGT | |
| | | | CCGAGCCTGTGGGGCCGACAGCTATGAGAT | |
| | | | GGAGGAAGACGGCGTCCGCAAGTGTAAGAA | |
| | | | GTGCGAAGGGCCTTGCCGCAAAG | |

### EXAMPLE 4

### Analytical Sensitivity of Gene Fusion Detection by a Two-Step PCR Target-Probe Hybridization Assay

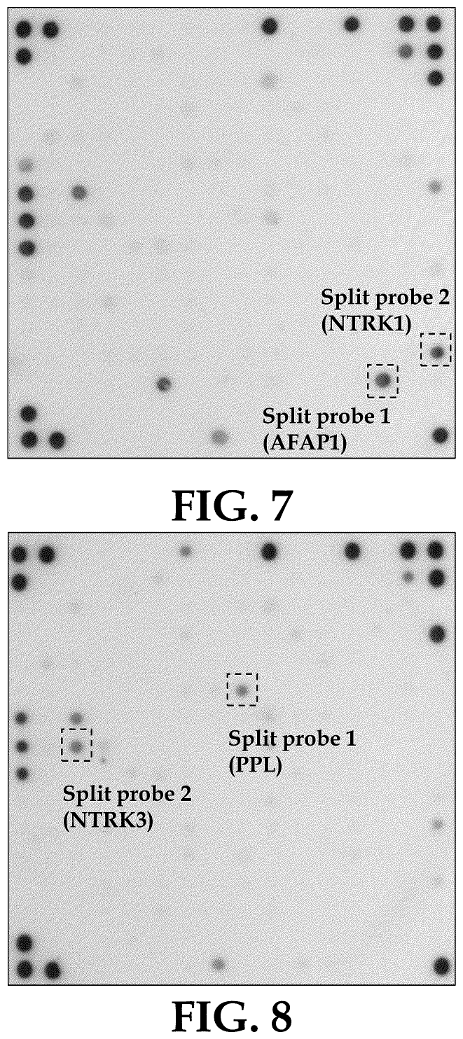

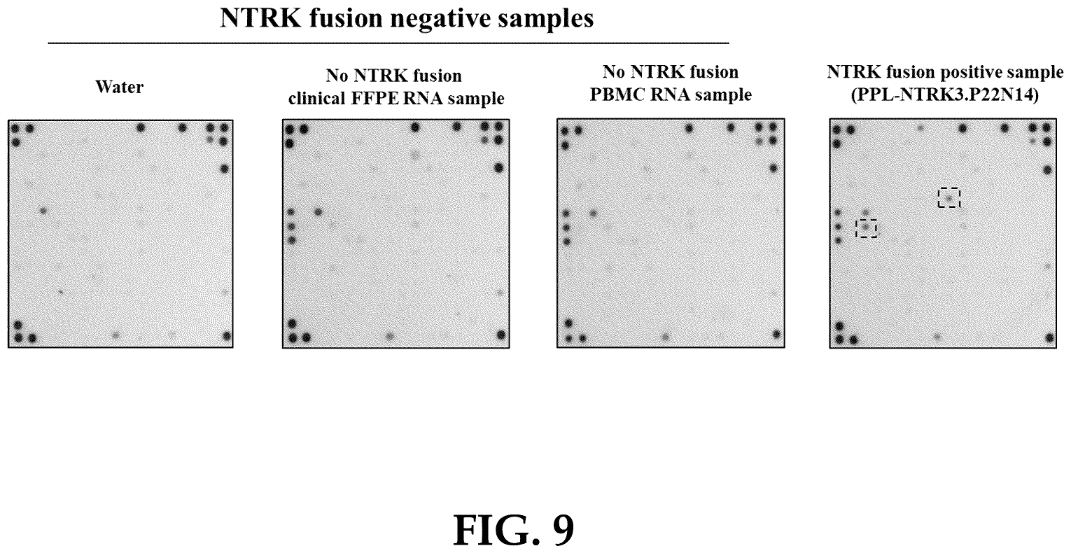

Before the analysis, the split probes targeting NTRK fusions are designed based on the nucleotide sequences of the fusion regions in the RNA transcript of the NTRK gene. To examine the analytical sensitivity of fusion probe assay, the DNA templates which include both known NTRK fusion types and previously unreported ones are synthesized (e.g. as shown in Table 1, Table 2, Table 10). There are a total of 165 synthetic DNA templates for known NTRK fusion types, and 50 synthetic DNA templates for novel NTRK fusion types. Each of the templates is diluted into 1,000 copies to examine the sensitivity of each fusion probe. Based on the detection range of each probe, the probe signaling has to be higher than its signaling threshold to be qualified at a certain analytical sensitivity. For known NTRK fusion types (e.g. as shown in FIG. 7) (total 165), the data showed that 85% (141) of NTRK fusion transcripts has sensitivity at 1000 copies. For novel NTRK fusion types (e.g. as shown in FIG. 8), the data showed that there are 98% (49) NTRK fusion transcripts have sensitivity at 1000 copies.

TABLE 10

| | Probe Sequence | |
|---|---|---|
| Probe Target | (from the 5′-end to the | |
| Region | the 3′-end) | SEQ ID NO |
| PPL (exon 22) | AATAAACCTGGTGGCTGGAG | 65 |
| NTRK3 (exon 14) | GTCATCAGTGGTGAGGAGGA | 66 |

### EXAMPLE 5

### Clinical Sample Verification of Fusion Detection by a Two-Step PCR Target-Probe Hybridization Assay

To verify the data analysis algorism, the 38 clinical FFPE-derived RNA samples have been analyzed by the ACTFusion panel (ACT Genomics Co., LTD) on a NGS platform. In a total of 38 thyroid cancer FFPE samples after data analysis, only one sample is NTRK fusion-positive, containing “ETV6-NTRK3” fusion. The test result of the NTRK fusion split probe assay is as shown in Table 11. This result is in concordance with NGS results by the ACTFusion panel. The test result of the NGS assay in the protocol of the ACTFusion panel is as shown in Table 12. The remaining 37 thyroid cancer FFPE samples are NTRK fusion negative and these samples are also negative in the NGS assay. This result showed 100% concordance between ACTFusion panel and split probe assay on these 38 clinical FFPE samples. Performance data of split probe chip assay which calculated with the result of sample is shown as NTRK fusion-positive or negative in Table 13.

TABLE 11

| Probe Target Region | Signal | Back Ground | Z-score |
|---|---|---|---|
| ETV6 (exon 4) | 103.7 | 38.2 | 1279.2 |
| NTRK3 (exon 14) | 102.2 | 35.9 | 1260.7 |
| NTRK3 (exon 14) | 89.3 | 40.4 | 1101.5 |
| ETV6 (exon 4) | 84.2 | 29.3 | 1038.5 |
| NTRK3 (exon 14) | 71.2 | 28.2 | 878.0 |

TABLE 12

| | | Number of | Number of consensus |
|---|---|---|---|
| Sample | | reads aligned | reads aligned |
| ID | Fusion transcript | to the target | to the target |
| 1 | ETV6-NTRK3.E4N14 | 22042 | 1785 |

TABLE 13

| | | Reference standard (NGS assay) | | |
|---|---|---|---|---|
| Performance | | Positive | Negative | Total |
| Test-result | Positive | 1 | 0 | 1 |
| | Negative | 0 | 37 | 37 |
| | Total | 1 | 37 | 38 |

Specificity=100.00% (95% Confidence Intervals: 90.51%-100.00%)
Accuracy =100.00% (95% Confidence Intervals: 90.75%-100.00%)
Positive percent agreement (PPA)=100% (95% Confidence Intervals: 2.5%-100.00%)
Positive predictive value (PPV)=100.00% (95% Confidence Intervals: 2.5%-100.00%).

### EXAMPLE 6

### Detection of BCR-ABL35INS Mutation by a Two-Step PCR Target-Probe Hybridization Assay

A two-step PCR target-probe hybridization assay is used here to detect BCR-ABL35INS mutation. This assay includes the same steps of obtaining RNA from a sample, reverse transcription of the RNA to obtain cDNA, PCR amplification of the BCR-ABL35INS mutation region of the cDNA (i.e., the target cDNA) using a BCR-ABL35INS mutation-specific primer pair to obtain a first amplified product of the target cDNA, PCR amplification of the first amplified product using a universal primer pair to obtain a second amplified product of the target cDNA, probe hybridization with the amplified target cDNA and detection of the probe-bound product.

### PCR Enrichment by Using a BCR-ABL35INS Mutation-Specific Primer Pair and a Universal Primer Pair

Each primer in the BCR-ABL35INS mutation-specific primer pair used in this assay is designed to have two segments. One segment, called an alternative splicing specific segment, is used to bind the 5′-end or the 3′-end of the BCR-ABL35INS mutation sequence. The alternative splicing specific segment may have the sequence of SEQ ID NO:67 or 68 (Table 14). The other segment, called a universal segment, encompasses the nucleotide sequence of the universal primer to be used in the second round of PCR. For alternative splicing specific PCR, the cDNA product is amplified according to the manufacturer's instructions, yielding a first amplified product and a second amplified product.

TABLE 14

| | Sequence of the BCR-ABL^{35INS} | | SEQ |
|---|---|---|---|
| Mutation | mutation-specific primer pair | | ID |
| type | (from the 5′ end to the 3′ end) | | NO |
| BCR-ABL^{35INS} | Forward | AGATGCTGACCAACTCG | 67 |
| alternative | Reverse | ACAATGTTCCAGGAATCCAG | 68 |
| splicing | | | |

### Probe Hybridization and Signal Detection

The second amplified product is denatured at 96° C. for 5 minutes and transferred to pre-blocked wells, each of which is printed with an array of probe spots, including the spots of the split probe (e.g. as shown in Table 1), the spots of control probes, and the spots of an anchor probe. The split probe is designed to bind to the sequence listed in Table 15 and the target-probe hybridization and signal detection are following our previous instructions.

The BCR-ABL35INS mutation and enrichment process have been previously reported (Yuda, Junichiro, et al. “Persistent detection of alternatively spliced BCR-ABL variant results in a failure to achieve deep molecular response.” Cancer science 108.11 (2017): 2204-2212.), incorporated herein by reference.

Since the BCR-ABL alternative splicing events give rise to mixed splice forms at mRNA levels, the identified approaches are often limited to working reliably at one design point. The approaches have in common that they probe or are designed to probe one splicing isoform at once, while the split probe assay can be used in probing several splicing isoforms (e.g. BCR-ABL or BCR-ABL35INS) and identify splice forms through signal analysis at once too. The efficiency of the split probe assay would increase because the same probe could be used in multiple splicing types.

It offers various advantages as a probing system including high precision and accuracy, a broad reportable range for novel mutation combinations, low cost, and ease of genetic manipulation.

TABLE 15

| | | | | Sequence of the fragment joining | SEQ |
|---|---|---|---|---|---|
| | | | | region | ID |
| Mutation type | Partner | Target | Another | (from the 5′ end to the 3′ end) | NO |
| BCR-ABL | BCR | ABL | ABL | ATGATGAGTCTCCGGGGCTCTATGGGT | 69 |
| alternative | exon 14 | exon 2-8 | intron 8 | TTCTGAATGTCATCGTCCACTCAGCCAC | |
| splicing | | | (35INS) | TGGATTTAAGCAGAGTTCAAAAGCCCT | |
| (BCR-ABL^{35INS}) | | | | TCAGCGGCCAGTAGCATCTGACTTTGA | |
| | | | | GCCTCAGGGTCTGAGTGAAGCCGCTCG | |
| | | | | TTGGAACTCCAAGGAAAACCTTCTCGC | |
| | | | | TGGACCCAGTGAAAATGACCCCAACCT | |
| | | | | TTTCGTTGCACTGTATGATTTTGTGGCC | |
| | | | | AGTGGAGATAACACTCTAAGCATAACT | |
| | | | | AAAG | |
| | | | | . . . | |
| | | | | CATTTGGAGTATTGCTTTGGGAAATTG | |
| | | | | CTACCTATGGCATGTCCCCTTACCCGGG | |
| | | | | AATTGACCTGTCCCAGGTGTATGAGCT | |
| | | | | GCTAGAGAAGGACTACCGCATGGAGC | |
| | | | | GCCCAGAAGGCTGCCCAGAGAAGGTC | |
| | | | | TATGAACTCATGCGAGCATACTTTGATA | |
| | | | | ACCGTGAAGAAAGAACAAGATAGAAG | |
| | | | | (partial DNA sequence represented | |
| | | | | here) | |

1. What is claimed is:
A method for detecting a DNA fragment joining event, comprising:
(a) obtaining a DNA or a DNA from an extracted RNA in a sample;
(b) enriching the DNA with a set of oligonucleotides to obtain a target nucleic acid;
(c) probing the target nucleic acid with a split probe comprising:
(i) a first split probe being complementary to the 3′ end of a partner DNA fragment, a second split probe being complementary to the 5′ end of a target DNA fragment, and/or a third split probe being complementary to a third DNA fragment, wherein a gap of the first and second split probes targeting sites on the target nucleic acid is within 0-80 bp; or
(ii) a first split probe being complementary to the 5′ end of a partner DNA fragment, a second split probe being complementary to the 3′ end of a target DNA fragment, and/or a third split probe being complementary to a third DNA fragment, wherein a gap of the first and second split probes targeting sites on the target nucleic acid is within 0-80 bp; and
(d) detecting a signal that reflects a binding between the split probe and the target nucleic acid.

2. The method of claim 1, wherein the set of oligonucleotides is a gene-specific primer or a gene-specific probe.

3. The method of claim 1, wherein the DNA is amplified by multiplex PCR with at least two pairs of a gene-specific primers in step (b).

4. The method of claim 3, further comprises:
(e) determining
(i) the partner DNA fragment as an upstream DNA fragment and/or the target DNA fragment as a downstream DNA fragment through confirming the signal based on the first split probe binding to the 3′ end of the partner DNA fragment and/or the second split probe binding to the 5′ end of the target DNA fragment;
(ii) the partner DNA fragment as a downstream DNA fragment and/or the target DNA fragment as an upstream DNA fragment through confirming the signal based on the first split probe binding to the 5′ end of the partner DNA fragment and/or the second split probe binding to the 3′ end of the target DNA fragment; or
(iii) whether or not the third DNA fragment is joined with the partner DNA fragment and the target DNA fragment through confirming the signal based on the third split probe binding to the third DNA fragment and a result of the target nucleic acid from an independent PCR.

5. The method of claim 3, wherein at least two pairs of the gene-specific primers are designed to obtain the target nucleic acid from the partner DNA fragment as an upstream DNA fragment.

6. The method of claim 3, wherein at least two pairs of the gene-specific primers are designed to obtain the target nucleic acid from the partner DNA fragment as a downstream DNA fragment.

7. The method of claim 3, wherein at least one of the gene-specific primers targets a DNA fragment joining boundary.

8. The method of claim 3, wherein the gene-specific primer targets within a distance of 0-80 bp from a DNA fragment joining boundary.

9. The method of claim 1, wherein the first split probe or the second split probe targets within a distance of 0-40 bp from a DNA fragment joining boundary.

10. The method of claim 1, wherein the first split probe is selected from the group consisting of SEQ ID Nos: 32, 35, and any complementary sequence thereof.

11. The method of claim 1, wherein the second split probe is selected from the group consisting of SEQ ID Nos: 33, 36, and any complementary sequence thereof.

12. The method of claim 1, wherein the third split probe is selected from the group consisting of SEQ ID Nos: 32, 33, 35, 36, and any complementary sequence thereof.

13. The method of claim 1, wherein a length of the split probe is 10-60 bp.

14. The method of claim 1, wherein in step (c), the target nucleic acid is probed with a split probe and a single probe targeting a DNA fragment joining boundary.

15. The method of claim 1, wherein the partner DNA fragment comprises a sequence of a partner gene selected from the group consisting of ACVR2A, AFAP1, AFF1, AGAP3, AGBL4, AGGF1, AKAP13, AKAP6, AKAP9, AMOTL2, ANKRD11, APIP, ARGLU1, ARHGEF11, ARHGEF2, ATG7, ATP1B, BAG4, BAIAP2L1, BCAN, BCL6, BCR, BICC1, BRD3, BRD4, BTBD1, CAPZA2, CBR4, CCDC170, CCDC6, CD74, CDK12, CDK5RAP2, CEL, CEP170, CFB, CHTOP, CLCN6, CLIP1, CLIP2, CLTC, CNIH4, CNTRL, COL25A1, COX5A, CPD, CREBBP, CTRC, CTTN, CUX1, CYSTM1, DAB2IP, DAZL, DCTN1, DLG1, DNAJC7, DNAJC8, EIF3E, ELL, EML1, EML4, ENO1, EPHB2, EPS15, ERC1, ESRP1, ETV6, EZR, FAM131B, FAT1, FCGRT, FGFR1, FGFR3, FIP1L1, FKBP10, FN1, FNDC3B, FRY, FUS, GKAP1, GOLGA4, GON4L, GOPC, GRB7, GRHL2, GRIPAP, GSE1, GTF2E2, GTF2IRD1, HACL1, HIP1, HNRNPA2B1, IKZF2, IKZF3, IQSEC1, IRF2BP2, JAK2, KANK1, KCTD16, KCTD8, KHDRBS1, KIAA1549, KIF5B, KRT20, KRT39, KRTAP1-4, KTN1, LIPI, LMNA, LMNTD1, LRRC71, LRRFIP1, LTBP4, LYN, MAD2L2, MAGI3, MBIP, MBNL1, MED1, MEF2D, MET, MIR548F1, MKRN1, MLLT1, MLLT10, MLLT11, MLLT3, MLLT4, MPRIP, MRPL24, MSN, MTSS1, MUC2, MYH9, MYO5A, NACC2, NAV1, NBPF20, NCOA4, NFASC, NOS1AP, NRG1, NRIP1, NTRK1, NTRK2, NTRK3, P2RX5, P2RY8, PAIP1, PAN3, PAPD7, PARN, PDE4DIP, PDGFRA, PDGFRB, PEAR1, PGAP3, PHC3, PHF20, PICALM, PLEKHA6, PML, POLD4, PPFIBP1, PPL, PPP1R1B, PRDM16, PRDX1, PRDX4, PRKAR1A, PRKAR1B, PRKAR2A, PRPSAP1, PSMB3, PTPRR, PTPRZ1, QKI, RAC1, RALGPS2, RANBP2, RBPMS, RET, RFWD2, RNF213, ROS1, RRBP1, SATB1, SCAF11, SCP2, SCYL3, SDC4, SEC31A, SEP6, SEP9, SHC1, SHKBP1, SIL1, SLC34A2, SLC39A11, SLC45A3, SLC4A4, SLMAP, SMIM18, SND1, SPECC1L, SPTBN1, SPTBN2, SQSTM1, SRCIN1, SRGAP3, SSBP2, STK11IP, STRN, STRN3, TACC3, TADA2A, TATDN1, TBC1D2, TBL1XR1, TFG, TIMP3, TKT, TLE4, TMEM106B, TMEM40, TMPRSS2, TNS3, TP53, TPM3, TPM4, TPR, TRAF2, TRAK1, TRIM24, TRIM33, TRIM4, TRIM63, UBE2D2, UBE2R2, UFD1, USP13, VANGL2, VCAN, VCL, VIM, VPS18, WHSC1L1, WIPF2, WNK2, XBP1, ZAN, ZBTB7B, ZNF710, and ZPR1.

16. The method of claim 1, wherein the target DNA fragment comprises a sequence of a target gene selected from the group consisting of ABL, AKT3, ALK, AXL, BCR, BRAF, CD74, ERBB2, ERBB4, ERG, ESR1, ETV1, ETV4, ETV5, ETV6, EZR, FGFR1, FGFR2, FGFR3, KIT, KMT2A, MET, NRG1, NRG2, NTRK1, NTRK2, NTRK3, NUTM1, PDGFRA, PDGFRB, PIK3CA, RAF1, RARA, RET, ROS1, RSPO2, SDC4, SLC34A2, and TMPRSS2.

17. The method of claim 1, wherein the partner DNA fragment and the target DNA fragment each comprises a different sequence from a same gene selected from the group consisting of AR, BCL2L1, BCL2L11, BCOR, BIN1, BRAF, BRCA1, BRCA2, CASP2, CD19, CD44, CXCR3, CCND1, DMP1, CDH1, EGFR, ER, EZH2, FAS, FGFR2, HRAS, IKZF1, KLF6, KRAS, MAP3K7, MCL1, MDM4, MET, MNK2, PIK3CD, PKM, RASGRP2, RON, RPS6KB, STAT3, TP53, TSC2, and VEGF.

18. The method of claim 1, wherein the DNA fragment joining event is selected from the group consisting of ACVR2A-AKT3, AFAP1-NTRK1, AFAP1-NTRK2, AFAP1-RET, AGAP3-BRAF, AGBL4-NTRK2, AGGF1-RAF1, AKAP13-NTRK3, AKAP13-RET, AKAP9-BRAF, AKT3-P2RX5, AKT3-PTPRR, AMOTL2-NTRK1, APIP-FGFR2, ARGLU1-NTRK1, ARHGEF11-NTRK1, ARHGEF2-NTRK1, ATG7-RAF1, ATP1B-NTRK1, AXL-MBIP, BAG4-FGFR1, BAIAP2L1-BRAF, BAIAP2L1-MET, BCAN-NTRK1, BCL6-RAF1, BCR-ABL, BCR-FGFR1, BCR-JAK2, BCR-NTRK2, BCR-RET, BRD3-NUTM1, BRD4-NUTM1, BTBD1-NTRK3, CAPZA2-MET, CBR4-ERBB4, CCDC6-BRAF, CCDC6-RET, CCDC6-ROS1, CD74-NRG1, CD74-NRG2, CD74-NTRK1, CD74-ROS1, CDK12-ERBB2, CDK5RAP2-BRAF, CEL-NTRK1, CEP170-AKT3, CHTOP-NTRK1, CLCN6-RAF1, CLIP1-ALK, CLIP1-ROS1, CLIP2-BRAF, CLIP2-MET, CLTC-ALK, CLTC-ROS1, CNTRL-KIT, COL25A1-ALK, COL25A1-FGFR2, COX5A-NTRK3, CPD-ERBB2, CTRC-NTRK1, CUX1-BRAF, CUX1-FGFR1, CUX1-RET, DCTN1-ALK, DCTN1-MET, DLG1-NTRK3, DNAJC8-ERBB2, EIF3E-RSPO2, EML1-NTRK2, EML4-ALK, EML4-BRAF, EML4-NTRK3, EML4-RET, EPHB2-NTRK1, EPS15-BRAF, EPS15-MET, EPS15-NTRK1, ERBB2-CDK12, ERBB2-CFB, ERBB2-CNIH4, ERBB2-CTTN, ERBB2-DNAJC7, ERBB2-ENO1, ERBB2-FCGRT, ERBB2-FKBP10, ERBB2-GRB7, ERBB2-GSE1, ERBB2-GTF2E2/SMIM18, ERBB2-IKZF3, ERBB2-KRT20, ERBB2-KRT39, ERBB2-KRTAP1-4, ERBB2-LMNTD1, ERBB2-LTBP4, ERBB2-MAD2L2, ERBB2-MED1, ERBB2-PARN, ERBB2-PGAP3, ERBB2-POLD4, ERBB2-PPP1R1B, ERBB2-PRDX4, ERBB2-PSMB3, ERBB2-SHKBP1, ERBB2-SLC39A11, ERBB2-SPTBN2, ERBB2-SRCIN1, ERBB2-TADA2A, ERBB2-TATDN1, ERBB2-XBP1, ERBB2-ZAN, ERBB4-AKAP6, ERBB4-FUS, ERBB4-IKZF2, ERBB4-STK11IP, ERC1-BRAF, ERC1-RET, ERC1-ROS1, ESRP1-RAF1, ESR1-CCDC170, ETV6-FGFR3, ETV6-NTRK2, ETV6-NTRK3, ETV6-PDGFRB, ETV6-PRDM16, EZR-ERBB4, EZR-ROS1, FAM131B-BRAF, FAT1-NTRK3, FGFR2-BICC1, FGFR2-TACC3, FGFR3-TACC3, FIP1L1-PDGFRA, FN1-ALK, FN1-ERBB4, FN1-FGFR1, FNDC3B-PIK3CA, FRY-NTRK3, GKAP1-NTRK2, GOLGA4-RAF1, GON4L-NTRK1, GOPC-ROS1, GRHL2-RSPO2, GRIPAP-NTRK1, GTF2IRD1-ALK, HACL1-RAF1, HIP1-ALK, HNRNPA2B1-NTRK3, IKZF2-ERBB4, IQSEC1-RAF1, IRF2BP2-NTRK1, KANK1-NTRK2, KCTD16-NTRK2, KCTD8-NTRK2, KHDRBS1-NTRK3, KIAA1549-BRAF, KIF5B-ALK, KIF5B-RET, KIF5B-ERBB4, KIT-ANKRD11, KIT-PDGFRA, KIT-SLC4A4, KMT2A-AFF1, KMT2A-CREBBP, KMT2A-DAB2IP, KMT2A-ELL, KMT2A-EPS15, KMT2A-MLLT1, KMT2A-MLLT10, KMT2A-MLLT11, KMT2A-MLLT3, KMT2A-MLLT4, KMT2A-SEP6, KMT2A-SEP9, KTN1-ALK, KTN1-RET, LIPI-NTRK1, LMNA-ALK, LMNA-NTRK1, LMNA-RAF1, LRRC71-NTRK1, LRRFIP1-FGFR1, LRRFIP1-MET, LYN-NTRK3, MAGI3-AKT3, MBNL1-RAF1, MEF2D-NTRK1, MET-MET, MIR548F1-NTRK1, MKRN1-BRAF, MPRIP-ALK, MPRIP-NTRK1, MPRIP-RAF1, MPRIP-RET, MRPL24-NTRK1, MSN-ALK, MSN-ROS1, MTSS1-ERBB2, MUC2-NTRK2, MYH9-ALK, MYO5A-NTRK3, MYO5A-ROS1, NACC2-NTRK2, NAV1-NTRK2, NBPF20-NTRK2, NCOA4-RET, NFASC-NTRK1, NOS1AP-NTRK1, NOS1AP-NTRK2, NRG2-CYSTM1, NRG2-UBE2D2, NRIP1-RSPO2, P2RY8-NTRK1, PAIP1-NTRK2, PAN3-NTRK2, PAPD7-RAF1, PDE4DIP-NTRK1, PEAR1-NTRK1, PHF20-NTRK1, PICALM-BRAF, PICALM-RET, PLEKHA6-NTRK1, PML-RARA, PPFIBP1-ALK, PPFIBP1-MET, PPFIBP1-ROS1, PPL-NTRK1, PRDX1-NTRK1, PRKAR1A-ALK, PRKAR1A-RET, PRKAR1B-ALK, PRKAR1B-BRAF, PRKAR2A-NTRK2, PRPSAP1-NTRK3, PTPRZ1-MET, QKI-NTRK2, QKI-RAF1, RAC1-AKT3, RAF1-ACTR2, RAF1-AGGF1, RAF1-DAZL, RAF1-ESRP1, RAF1-PHC3, RAF1-TMEM40, RAF1-TRAK1, RAF1-ZPR1, RALGPS2-NTRK3, RANBP2-ALK, RANBP2-FGFR1, RBPMS-NTRK3, RFWD2-NTRK1, RNF213-ALK, RNF213-NTRK1, RRBP1-ALK, RRBP1-RET, SATB1-ALK, SATB1-RET, SCAF11-PDGFRA, SCP2-NTRK1, SCYL3-NTRK1, SDC4-NRG1, SDC4-ROS1, SEC31A-ALK, SHC1-ERBB2, SIL1-NRG2, SLC34A2-MET, SLC34A2-ROS1, SLC45A3-BRAF, SLC45A3-ERG, SLC45A3-FGFR2, SLMAP-NTRK2, SND1-BRAF, SPECC1L-NTRK2, SPECC1L-NTRK3, SPTBN1-ALK, SQSTM1-ALK, SQSTM1-FGFR1, SQSTM1-NTRK1, SQSTM1-NTRK2, SQSTM1-NTRK3, SRGAP3-RAF1, SRGAP3-SRGAP3-RAF1, SSBP2-NTRK1, STRN-ALK, STRN-NTRK2, STRN-NTRK3, STRN3-BRAF, STRN3-NTRK1, STRN3-NTRK2, STRN3-NTRK3, TBC1D2-NTRK2, TBL1XR1-NRG1, TBL1XR1-PIK3CA, TBL1XR1-RET, TFG-ALK, TFG-MET, TFG-NTRK1, TFG-NTRK3, TFG-RET, TFG-ROS1, TIMP3-ALK, TIMP3-NTRK1, TKT-ERBB2, TLE4-NTRK2, TMEM106B-BRAF, TMEM106B-ROS1, TMPRSS2-ERG, TMPRSS2-ETV1, TMPRSS2-ETV4, TMPRSS2-ETV5, TNS3-NTRK2, TP53-NTRK1, TPM3-ALK, TPM3-NTRK1, TPM3-ROS1, TPM4-ALK, TPM4-NTRK3, TPR-ALK, TPR-BRAF, TPR-FGFR1, TPR-MET, TPR-NTRK1, TRAF2-NTRK2, TRAK1-RAF1, TRIM24-BRAF, TRIM24-FGFR1, TRIM24-NTRK2, TRIM24-RET, TRIM33-RET, TRIM33-NTRK1, TRIM4-BRAF, TRIM4-MET, TRIM63-NTRK1, UBE2R2-NTRK3, UFD1-NTRK2, USP13-PIK3CA, VANGL2-NTRK1, VCAN-NTRK2, VCL-ALK, VCL-NTRK2, VIM-NTRK3, VPS18-NTRK3, WHSC1L1-FGFR1, WHSC1L1-NUTM1, WIPF2-ERBB2, WNK2-NTRK2, ZBTB7B-NTRK1, and ZNF710-NTRK3 mutations.

19. The method of claim 1, wherein the third DNA fragment comprises a sequence of a partner gene or a target gene.

20. The method of claim 1, wherein in step (b), the DNA is amplified with a gene-specific primer first and subsequently with a universal primer to obtain the target nucleic acid.

21. The method of claim 1, wherein the signal is selected from the group consisting of dyes, chemiluminescent dyes, fluorescent molecules, radioisotopes, spin labels, enzymes, haptens, quantum dots, beads, aminohexyls, and pyrenes.

22. A method for distinguishing an alternative splicing event, comprising:
(a) probing a target nucleic acid with a split probe, comprising:
(i) a first split probe being complementary to the 3′ end of a partner DNA fragment, a second split probe being complementary to the 5′ end of a target DNA fragment, and/or a third split probe being complementary to a third DNA fragment, wherein a gap of the first and second split probes targeting sites on the target nucleic acid is within 0-80 bp; or
(ii) a first split probe being complementary to the 5′ end of a partner DNA fragment, a second split probe being complementary to the 3′ end of a target DNA fragment, and/or a third split probe being complementary to a third DNA fragment, wherein a gap of the first and second split probes targeting sites on the target nucleic acid is within 0-80 bp;
(b) detecting a signal that reflects a binding between the split probe and the target nucleic acid;
(c) determining
(i) the partner DNA fragment as an upstream DNA fragment and/or the target DNA fragment as a downstream DNA fragment through confirming the signal based on the first split probe binding to the 3′ end of the partner DNA fragment and/or the second split probe binding to the 5′ end of the target DNA fragment;
(ii) the partner DNA fragment as a downstream DNA fragment and/or the target DNA fragment as an upstream DNA fragment through confirming the signal based on the first split probe binding to the 5′ end of the partner DNA fragment and/or the second split probe binding to the 3′ end of the target DNA fragment, or
(iii) the third DNA fragment that is joined with the partner DNA fragment and the target DNA fragment through confirming the signal based on the third split probe binding to the third DNA fragment; and
(d) comparing whether a length of the target nucleic acid is identical to a reference sequence.

23. The method of claim 22, wherein the target nucleic acid is amplified by a set of oligonucleotides.

24. The method of claim 22, wherein the target nucleic acid is amplified by multiplex PCR with at least two pairs of a gene-specific primers.

25. The method of claim 24, further comprises:
(e) reconfirming by an independent PCR.

26. The method of claim 24, wherein at least two pairs of the gene-specific primers are designed to obtain the target nucleic acid from the partner DNA fragment as an upstream DNA fragment.

27. The method of claim 24, wherein at least two pairs of the gene-specific primers are designed to obtain the target nucleic acid from the partner DNA fragment as a downstream DNA fragment.

28. The method of claim 24, wherein at least one of the gene-specific primers targets a DNA fragment joining boundary.

29. The method of claim 24, wherein the gene-specific primers targets within a distance of 0-80 bp from a DNA fragment joining boundary.

30. The method of claim 24, wherein a product of the multiplex PCR is amplified subsequently with a universal primer to obtain the target nucleic acid.

31. The method of claim 22, wherein a distance between the first and second split probes targeting site and a DNA fragment joining boundary is within 0-40 bp.

32. The method of claim 22, wherein a length of the split probe is 10-60 bp.

33. The method of claim 22, wherein in step (a), the target nucleic acid is probed with a split probe and a single probe targeting a DNA fragment joining boundary.

34. The method of claim 22, wherein the partner DNA fragment and the target DNA fragment each comprises a different sequence of a same gene selected from the group consisting of AR, BCL2L1, BCL2L11, BCOR, BIN1, BRAF, BRCA1, BRCA2, CASP2, CD19, CD44, CXCR3, CCND1, DMP1, CDH1, EGFR, ER, EZH2, FAS, FGFR2, HRAS, IKZF1, KLF6, KRAS, MAP3K7, MCL1, MDM4, MET, MNK2, PIK3CD, PKM, RASGRP2, RON, RPS6KB, STAT3, TP53, TSC2, and VEGF.

35. The method of claim 22, wherein the alternative splicing event is BCR-ABL mutation.

36. The method of claim 22, wherein the third DNA fragment comprises a sequence of a partner gene or a target gene.

37. The method of claim 22, wherein the signal is selected from the group consisting of dyes, chemiluminescent dyes, fluorescent molecules, radioisotopes, spin labels, enzymes, haptens, quantum dots, beads, aminohexyls, and pyrenes.

38. A method for treating a subject, comprising:
(a) determining whether a subject is at risk of cancer or a genotype, comprising detecting a DNA fragment joining event by the method of claim 1 and/or distinguishing an alternative splicing event by the method of claim 22 of a sample from the subject; and
(b) administering
(i) a therapeutically effective amount of a siRNA targeting the DNA fragment joining event and/or the alternative splicing event;
(ii) a therapeutically effective amount of an inhibitor of a fusion protein encoded by the DNA fragment joining event and/or the alternative splicing event;
(iii) a therapeutically effective amount of an agent that inhibits a fusion protein encoded by the DNA fragment joining event and/or the alternative splicing event;
(iv) a therapeutically effective amount of an anticancer agent selected from the group consisting of cytokines, apoptosis-inducing agents, anti-angiogenic agents, chemotherapeutic agents, radio-therapeutic agents, and anticancer immunotoxins; or
(v) providing a targeted genome editing procedure within cells of the subject.

39. The method of claim 38, wherein the DNA fragment joining event presents with a sequence of a partner gene selected from the group consisting of ACVR2A, AFAP1, AFF1, AGAP3, AGBL4, AGGF1, AKAP13, AKAP6, AKAP9, AMOTL2, ANKRD11, APIP, ARGLU1, ARHGEF11, ARHGEF2, ATG7, ATP1B, BAG4, BAIAP2L1, BCAN, BCL6, BCR, BICC1, BRD3, BRD4, BTBD1, CAPZA2, CBR4, CCDC170, CCDC6, CD74, CDK12, CDK5RAP2, CEL, CEP170, CFB, CHTOP, CLCN6, CLIP1, CLIP2, CLTC, CNIH4, CNTRL, COL25A1, COX5A, CPD, CREBBP, CTRC, CTTN, CUX1, CYSTM1, DAB2IP, DAZL, DCTN1, DLG1, DNAJC7, DNAJC8, EIF3E, ELL, EML1, EML4, ENO1, EPHB2, EPS15, ERC1, ESRP1, ETV6, EZR, FAM131B, FAT1, FCGRT, FGFR1, FGFR3, FIP1L1, FKBP10, FN1, FNDC3B, FRY, FUS, GKAP1, GOLGA4, GON4L, GOPC, GRB7, GRHL2, GRIPAP, GSE1, GTF2E2, GTF2IRD1, HACL1, HIP1, HNRNPA2B1, IKZF2, IKZF3, IQSEC1, IRF2BP2, JAK2, KANK1, KCTD16, KCTD8, KHDRBS1, KIAA1549, KIF5B, KRT20, KRT39, KRTAP1-4, KTN1, LIPI, LMNA, LMNTD1, LRRC71, LRRFIP1, LTBP4, LYN, MAD2L2, MAGI3, MBIP, MBNL1, MED1, MEF2D, MET, MIR548F1, MKRN1, MLLT1, MLLT10, MLLT11, MLLT3, MLLT4, MPRIP, MRPL24, MSN, MTSS1, MUC2, MYH9, MYO5A, NACC2, NAV1, NBPF20, NCOA4, NFASC, NOS1AP, NRG1, NRIP1, NTRK1, NTRK2, NTRK3, P2RX5, P2RY8, PAIP1, PAN3, PAPD7, PARN, PDE4DIP, PDGFRA, PDGFRB, PEAR1, PGAP3, PHC3, PHF20, PICALM, PLEKHA6, PML, POLD4, PPFIBP1, PPL, PPP1R1B, PRDM16, PRDX1, PRDX4, PRKAR1A, PRKAR1B, PRKAR2A, PRPSAP1, PSMB3, PTPRR, PTPRZ1, QKI, RAC1, RALGPS2, RANBP2, RBPMS, RET, RFWD2, RNF213, ROS1, RRBP1, SATB1, SCAF11, SCP2, SCYL3, SDC4, SEC31A, SEP6, SEP9, SHC1, SHKBP1, SIL1, SLC34A2, SLC39A11, SLC45A3, SLC4A4, SLMAP, SMIM18, SND1, SPECC1L, SPTBN1, SPTBN2, SQSTM1, SRCIN1, SRGAP3, SSBP2, STK11IP, STRN, STRN3, TACC3, TADA2A, TATDN1, TBC1D2, TBL1XR1, TFG, TIMP3, TKT, TLE4, TMEM106B, TMEM40, TMPRSS2, TNS3, TP53, TPM3, TPM4, TPR, TRAF2, TRAK1, TRIM24, TRIM33, TRIM4, TRIM63, UBE2D2, UBE2R2, UFD1, USP13, VANGL2, VCAN, VCL, VIM, VPS18, WHSC1L1, WIPF2, WNK2, XBP1, ZAN, ZBTB7B, ZNF710, and ZPR1.

40. The method of claim 38, wherein the DNA fragment joining event presents with a sequence of a target gene selected from the group consisting of ABL, AKT3, ALK, AXL, BCR, BRAF, CD74, ERBB2, ERBB4, ERG, ESR1, ETV1, ETV4, ETV5, ETV6, EZR, FGFR1, FGFR2, FGFR3, KIT, KMT2A, MET, NRG1, NRG2, NTRK1, NTRK2, NTRK3, NUTM1, PDGFRA, PDGFRB, PIK3CA, RAF1, RARA, RET, ROS1, RSPO2, SDC4, SLC34A2, and TMPRSS2.

41. The method of claim 38, wherein the alternative splicing event presents with a different sequence of a same gene selected from the group consisting of AR, BCL2L1, BCL2L11, BCOR, BIN1, BRAF, BRCA1, BRCA2, CASP2, CD19, CD44, CXCR3, CCND1, DMP1, CDH1, EGFR, ER, EZH2, FAS, FGFR2, HRAS, IKZF1, KLF6, KRAS, MAP3K7, MCL1, MDM4, MET, MNK2, PIK3CD, PKM, RASGRP2, RON, RPS6KB, STAT3, TP53, TSC2, and VEGF.

42. The method of claim 38, wherein the DNA fragment joining event or the alternative splicing event is BCR-ABL mutation.

43. The method of claim 38, wherein the alternative splicing event is selected from the group consisting of constitutive splicing, exon skipping, intron retention, mutually exclusive exons, and alternative 5′ or 3′ splice site.

44. The method of claim 38, wherein the cancer is selected from the group consisting of carcinoma, sarcoma, lymphoma, leukemia, and myeloma.

45. The method of claim 38, wherein the cancer is selected from the group consisting of brain cancer, breast cancer, colon cancer, endocrine gland cancer, esophageal cancer, female reproductive organ cancer, head and neck cancer, hepatobiliary system cancer, kidney cancer, lung cancer, mesenchymal cell neoplasm, prostate cancer, skin cancer, stomach cancer, tumor of the exocrine pancreas, and urinary system cancer.

46. A kit for detecting a sample with a DNA fragment joining event and/or an alternative splicing event, comprising:
(a) a set of oligonucleotides;
(b) a split probe, comprising:
(i) a first split probe being complementary to the 3′ end of a partner DNA fragment, a second split probe being complementary to the 5′ end of a target DNA fragment, and/or a third split probe being complementary to a third DNA fragment, wherein a gap of the first and second split probes targeting sites on a target nucleic acid is within 0-80 bp; or
(ii) a first split probe being complementary to the 5′ end of a partner DNA fragment, a second split probe being complementary to the 3′ end of a target DNA fragment, and/or a third split probe being complementary to a third DNA fragment, wherein a gap of the first and second split probes targeting sites on a target nucleic acid is within 0-80 bp; and
(c) a probe hybridization assay for detecting a split probe hybridization signal, for which comprises dyes, chemiluminescent dyes, fluorescent molecule, radioisotopes, spin labels, enzymes, haptens, quantum dots, beads, aminohexyls, and pyrenes.

47. The kit of claim 46, wherein the set of oligonucleotides is a gene-specific primer or a gene-specific probe.

48. The kit of claim 46, wherein the kit comprises at least two pairs of a gene-specific primers.

49. The kit of claim 48, wherein the gene-specific primer is designed to obtain the target nucleic acid from the partner DNA fragment as an upstream DNA fragment.

50. The kit of claim 48, wherein the gene-specific primer is designed to obtain the target nucleic acid from the partner DNA fragment as a downstream DNA fragment.

51. The kit of claim 46, further comprises a universal primer.

52. The kit of claim 48, wherein at least one of the gene-specific primers targets a DNA fragment joining boundary.

53. The kit of claim 48, wherein the gene-specific primers targets within a distance of 0-80 bp from a DNA fragment joining boundary.

54. The kit of claim 46, wherein the first split probe or the second split probe targets within a distance of 0-40 bp from a DNA fragment joining boundary.

55. The kit of claim 46, wherein the first split probe is selected from the group consisting of SEQ ID Nos: 32, 35, and any complementary sequence thereof.

56. The kit of claim 46, wherein the second split probe is selected from the group consisting of SEQ ID Nos: 33, 36, and any complementary sequence thereof.

57. The kit of claim 46, wherein the third split probe is selected from the group consisting of SEQ ID Nos: 32, 33, 35, 36, and any complementary sequence thereof.

58. The kit of claim 46, wherein a length of the split probe is 10-60 bp.

59. The kit of claim 46, further comprises a single probe targeting a DNA fragment joining boundary.

60. The kit of claim 46, wherein the first split probe is complementary to the partner DNA fragment including a sequence of a partner gene selected from the group consisting of ACVR2A, AFAP1, AFF1, AGAP3, AGBL4, AGGF1, AKAP13, AKAP6, AKAP9, AMOTL2, ANKRD11, APIP, ARGLU1, ARHGEF11, ARHGEF2, ATG7, ATP1B, BAG4, BAIAP2L1, BCAN, BCL6, BCR, BICC1, BRD3, BRD4, BTBD1, CAPZA2, CBR4, CCDC170, CCDC6, CD74, CDK12, CDK5RAP2, CEL, CEP170, CFB, CHTOP, CLCN6, CLIP1, CLIP2, CLTC, CNIH4, CNTRL, COL25A1, COX5A, CPD, CREBBP, CTRC, CTTN, CUX1, CYSTM1, DAB2IP, DAZL, DCTN1, DLG1, DNAJC7, DNAJC8, EIF3E, ELL, EML1, EML4, ENO1, EPHB2, EPS15, ERC1, ESRP1, ETV6, EZR, FAM131B, FAT1, FCGRT, FGFR1, FGFR3, FIP1L1, FKBP10, FN1, FNDC3B, FRY, FUS, GKAP1, GOLGA4, GON4L, GOPC, GRB7, GRHL2, GRIPAP, GSE1, GTF2E2, GTF2IRD1, HACL1, HIP1, HNRNPA2B1, IKZF2, IKZF3, IQSEC1, IRF2BP2, JAK2, KANK1, KCTD16, KCTD8, KHDRBS1, KIAA1549, KIF5B, KRT20, KRT39, KRTAP1-4, KTN1, LIPI, LMNA, LMNTD1, LRRC71, LRRFIP1, LTBP4, LYN, MAD2L2, MAGI3, MBIP, MBNL1, MED1, MEF2D, MET, MIR548F1, MKRN1, MLLT1, MLLT10, MLLT11, MLLT3, MLLT4, MPRIP, MRPL24, MSN, MTSS1, MUC2, MYH9, MYO5A, NACC2, NAV1, NBPF20, NCOA4, NFASC, NOS1AP, NRG1, NRIP1, NTRK1, NTRK2, NTRK3, P2RX5, P2RY8, PAIP1, PAN3, PAPD7, PARN, PDE4DIP, PDGFRA, PDGFRB, PEAR1, PGAP3, PHC3, PHF20, PICALM, PLEKHA6, PML, POLD4, PPFIBP1, PPL, PPP1R1B, PRDM16, PRDX1, PRDX4, PRKAR1A, PRKAR1B, PRKAR2A, PRPSAP1, PSMB3, PTPRR, PTPRZ1, QKI, RAC1, RALGPS2, RANBP2, RBPMS, RET, RFWD2, RNF213, ROS1, RRBP1, SATB1, SCAF11, SCP2, SCYL3, SDC4, SEC31A, SEP6, SEP9, SHC1, SHKBP1, SIL1, SLC34A2, SLC39A11, SLC45A3, SLC4A4, SLMAP, SMIM18, SND1, SPECC1L, SPTBN1, SPTBN2, SQSTM1, SRCIN1, SRGAP3, SSBP2, STK11IP, STRN, STRN3, TACC3, TADA2A, TATDN1, TBC1D2, TBL1XR1, TFG, TIMP3, TKT, TLE4, TMEM106B, TMEM40, TMPRSS2, TNS3, TP53, TPM3, TPM4, TPR, TRAF2, TRAK1, TRIM24, TRIM33, TRIM4, TRIM63, UBE2D2, UBE2R2, UFD1, USP13, VANGL2, VCAN, VCL, VIM, VPS18, WHSC1L1, WIPF2, WNK2, XBP1, ZAN, ZBTB7B, ZNF710, and ZPR1.

61. The kit of claim 46, wherein the second split probe is complementary to the target DNA fragment including a sequence of a target gene selected from the group consisting of ABL, AKT3, ALK, AXL, BCR, BRAF, CD74, ERBB2, ERBB4, ERG, ESR1, ETV1, ETV4, ETV5, ETV6, EZR, FGFR1, FGFR2, FGFR3, KIT, KMT2A, MET, NRG1, NRG2, NTRK1, NTRK2, NTRK3, NUTM1, PDGFRA, PDGFRB, PIK3CA, RAF1, RARA, RET, ROS1, RSPO2, SDC4, SLC34A2, and TMPRSS2.

62. The kit of claim 46, wherein the first split probe and the second split probe are complementary to the partner DNA fragment and the target DNA fragment each including a different sequence of a same gene selected from the group consisting of AR, BCL2L1, BCL2L11, BCOR, BIN1, BRAF, BRCA1, BRCA2, CASP2, CD19, CD44, CXCR3, CCND1, DMP1, CDH1, EGFR, ER, EZH2, FAS, FGFR2, HRAS, IKZF1, KLF6, KRAS, MAP3K7, MCL1, MDM4, MET, MNK2, PIK3CD, PKM, RASGRP2, RON, RPS6KB, STAT3, TP53, TSC2, and VEGF.

63. The kit of claim 46, wherein the DNA fragment joining event or the alternative splicing event is BCR-ABL mutation.

64. The kit of claim 46, wherein the third split probe is complementary to the third DNA fragment including a sequence of a partner gene or a target gene.


SEQUENCE LISTING (69 sequences)

SEQ ID NO: 1 - DNA, 20 bp, Artificial Sequence
  ABL (exon 2)
gcggccagtagcatctgact

SEQ ID NO: 2 - DNA, 20 bp, Artificial Sequence
  ABL (intron 8)
tgataaccgtgaagaaagaa

SEQ ID NO: 3 - DNA, 20 bp, Artificial Sequence
  AFAP1 (exon 4)
ctccggaatacatcacatca

SEQ ID NO: 4 - DNA, 24 bp, Artificial Sequence
  ALK (exon 20)
tgtaccgccggaagcaccaggagc

SEQ ID NO: 5 - DNA, 18 bp, Artificial Sequence
  AR (exon 3)
gcaattgcaagcatctca

SEQ ID NO: 6 - DNA, 33 bp, Artificial Sequence
  AR (exon 4)
aggaaaaattgtccatcttgtcgtcttcggaaa

SEQ ID NO: 7 - DNA, 29 bp, Artificial Sequence
  AXL (exon 20)
tacagagtgtggactctggtgcctccaga

SEQ ID NO: 8 - DNA, 20 bp, Artificial Sequence
  BAG4 (exon 2)
catatcctgtaagaccagaa

SEQ ID NO: 9 - DNA, 25 bp, Artificial Sequence
  BCR (exon 14)
gccactggatttaagcagagttcaa

SEQ ID NO: 10 - DNA, 25 bp, Artificial Sequence
  BICC1 (exon 2)
tgctgctgaagggaaaggcagaagt

SEQ ID NO: 11 - DNA, 29 bp, Artificial Sequence
  CCDC6 (exon 1)
gaaccgcgacctgcgcaaagccagcgtga

SEQ ID NO: 12 - DNA, 24 bp, Artificial Sequence
  EGFR (exon 1)
gtcgggctctggaggaaaagaaag

SEQ ID NO: 13 - DNA, 20 bp, Artificial Sequence
  EGFR (exon 8)
gtaattatgtggtgacagat

SEQ ID NO: 14 - DNA, 19 bp, Artificial Sequence
  EML4 (exon13)
cacctgggaaaggacctaa

SEQ ID NO: 15 - DNA, 29 bp, Artificial Sequence
  FGFR1 (exon 6)
actctgtggtgccctctgacaagggcaac

SEQ ID NO: 16 - DNA, 24 bp, Artificial Sequence
  FGFR2 (exon 10)
ttacagcttccccagactacctgg

SEQ ID NO: 17 - DNA, 18 bp, Artificial Sequence
  FGFR2 (exon 17)
attctcactctcacaacc

SEQ ID NO: 18 - DNA, 18 bp, Artificial Sequence
  FGFR2 (exon 8)
accagtctgcctggctca

SEQ ID NO: 19 - DNA, 24 bp, Artificial Sequence
  FGFR3 (exon 17)
tgtccttaccgtgacgtccaccga

SEQ ID NO: 20 - DNA, 31 bp, Artificial Sequence
  MBIP (exon 4)
aagctgaaatcaatgaaaacaacgtcaggga

SEQ ID NO: 21 - DNA, 30 bp, Artificial Sequence
  MET (exon 13)
tgtggctgaaaaagagaaagcaaattaaag

SEQ ID NO: 22 - DNA, 20 bp, Artificial Sequence
  MET (exon 15)
atcagtttcctaattcatct

SEQ ID NO: 23 - DNA, 20 bp, Artificial Sequence
  NTRK1 (exon 10)
acagcacatctggagacccg

SEQ ID NO: 24 - DNA, 22 bp, Artificial Sequence
  PDGFRA (exon 2)
tttcccagagctatggggactt

SEQ ID NO: 25 - DNA, 20 bp, Artificial Sequence
  PPL (exon 22)
aataaacctggtggctggag

SEQ ID NO: 26 - DNA, 22 bp, Artificial Sequence
  RET (exon 12)
aagtgggaattccctcggaaga

SEQ ID NO: 27 - DNA, 20 bp, Artificial Sequence
  ROS1 (exon 32)
tcccaaattactagaaggga

SEQ ID NO: 28 - DNA, 17 bp, Artificial Sequence
  SCAF11 (exon 1)
cctcgacctcggtctga

SEQ ID NO: 29 - DNA, 24 bp, Artificial Sequence
  SLC34A2 (exon 4)
tcgtgtgctccctggatattctta

SEQ ID NO: 30 - DNA, 19 bp, Artificial Sequence
  TACC3 (exon 8)
agtcggccttgaggaagca

SEQ ID NO: 31 - DNA, 20 bp, Artificial Sequence
  NTRK
gggagaatagcaggtcccgt

SEQ ID NO: 32 - DNA, 20 bp, Artificial Sequence
  ETV6 (exon 5)
cgccatgcccattgggagaa

SEQ ID NO: 33 - DNA, 20 bp, Artificial Sequence
  NTRK3 (exon 14)
gtcccgtggctgtcatcagt

SEQ ID NO: 34 - DNA, 20 bp, Artificial Sequence
  NTRK
tggtgtattaggcccagcct

SEQ ID NO: 35 - DNA, 20 bp, Artificial Sequence
  QKI (exon 6)
tatcctattgaacctagtgg

SEQ ID NO: 36 - DNA, 20 bp, Artificial Sequence
  NTRK2 (exon 16)
gcccagcctccgttatcagc

SEQ ID NO: 37 - DNA, 323 bp, Artificial Sequence
  MET-MET alternative splicing
tggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggc

SEQ ID NO: 38 - DNA, 26 bp, Artificial Sequence
  MET-MET alternative splicing Foward primer
aagagaaagcaaattaaagatcagtt

SEQ ID NO: 39 - DNA, 19 bp, Artificial Sequence
  MET-MET alternative splicing Reverse primer
ctgtcagaggatactgcac

SEQ ID NO: 40 - DNA, 19 bp, Artificial Sequence
  Universal primer No. U01
gttttcccagtcacgacgt

SEQ ID NO: 41 - DNA, 21 bp, Artificial Sequence
  Universal primer No. U02
gcaaatggcattctgacatcc

SEQ ID NO: 42 - DNA, 22 bp, Artificial Sequence
  Universal primer No. U03
gcggataacaatttcacacagg

SEQ ID NO: 43 - DNA, 17 bp, Artificial Sequence
  Universal primer No. U04
cgtccatgccgagagtg

SEQ ID NO: 44 - DNA, 23 bp, Artificial Sequence
  Universal primer No. U05
ctttatgtttttggcgtcttcca

SEQ ID NO: 45 - DNA, 21 bp, Artificial Sequence
  Universal primer No. U06
gactggttccaattgacaagc

SEQ ID NO: 46 - DNA, 19 bp, Artificial Sequence
  Universal primer No. U07
gcgtgaatgtaagcgtgac

SEQ ID NO: 47 - DNA, 18 bp, Artificial Sequence
  Universal primer No. U08
tgtaaaacgacggccagt

SEQ ID NO: 48 - DNA, 20 bp, Artificial Sequence
  Universal primer No. U09
aagggtcttgcgaaggatag

SEQ ID NO: 49 - DNA, 23 bp, Artificial Sequence
  Universal primer No. U10
gggttatgctagttattgctcag

SEQ ID NO: 50 - DNA, 20 bp, Artificial Sequence
  ETV6-NTRK3 fusion Forward primer
ccacatcatggtctctgtct

SEQ ID NO: 51 - DNA, 17 bp, Artificial Sequence
  ETV6-NTRK3 fusion Reverse primer
tggttgatgtggtgcag

SEQ ID NO: 52 - DNA, 19 bp, Artificial Sequence
  TFG-NTRK1 fusion Forward primer
acagcagccaccatataca

SEQ ID NO: 53 - DNA, 17 bp, Artificial Sequence
  TFG-NTRK1 fusion Reverse primer
agaccccaaaaggtgtt

SEQ ID NO: 54 - DNA, 30 bp, Artificial Sequence
  TFG-NTRK1 fusion Foward primer
atcctttaaaaaaccaagatgaaatcaata

SEQ ID NO: 55 - DNA, 17 bp, Artificial Sequence
  TFG-NTRK1 fusion Forward primer
gagaaggggatgcacca

SEQ ID NO: 56 - DNA, 17 bp, Artificial Sequence
  TPM3-NTRK1 fusion Forward primer
gacccgtgctgagtttg

SEQ ID NO: 57 - DNA, 17 bp, Artificial Sequence
  TPM3-NTRK1 fusion Reverse primer
aaatgcagggacatggc

SEQ ID NO: 58 - DNA, 22 bp, Artificial Sequence
  ETV6-NTRK2 fusion Forward primer
ttccaccctggaaactctatac

SEQ ID NO: 59 - DNA, 21 bp, Artificial Sequence
  ETV6-NTRK2 fusion Reverse primer
cattggagatgtgatggagtg

SEQ ID NO: 60 - DNA, 19 bp, Artificial Sequence
  TFG-NTRK3 fusion Forward primer
acagcagccaccatataca

SEQ ID NO: 61 - DNA, 17 bp, Artificial Sequence
  TFG-NTRK3 fusion Reverse primer
ctcgatgcagtgctcca

SEQ ID NO: 62 - DNA, 19 bp, Artificial Sequence
  EGFRvIII mutation-specific forward primer
gggctctggaggaaaagaa

SEQ ID NO: 63 - DNA, 19 bp, Artificial Sequence
  EGFRvIII mutation-specific reverse primer
tccatctcatagctgtcgg

SEQ ID NO: 64 - DNA, 328 bp, Artificial Sequence
  EGFR-EGFR alternative splicing
ccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaaggtaattatgtggtgacagatcacggctcgtgcgtccgagcctgtggggccgacagctatgagatggaggaagacggcgtccgcaagtgtaagaagtgcgaagggccttgccgcaaag

SEQ ID NO: 65 - DNA, 20 bp, Artificial Sequence
  PPL (exon 22) Probe Sequence
aataaacctggtggctggag

SEQ ID NO: 66 - DNA, 20 bp, Artificial Sequence
  NTRK3 (exon 14) Probe Sequence
gtcatcagtggtgaggagga

SEQ ID NO: 67 - DNA, 17 bp, Artificial Sequence
  BCR-ABL35INS mutation-specific forward primer
agatgctgaccaactcg

SEQ ID NO: 68 - DNA, 20 bp, Artificial Sequence
  BCR-ABL35INS mutation-specific reverse primer
acaatgttccaggaatccag

SEQ ID NO: 69 - DNA, 1454 bp, Artificial Sequence
  BCR-ABL alternative splicing (BCR-ABL35INS)
atgatgagtctccggggctctatgggtttctgaatgtcatcgtccactcagccactggatttaagcagagttcaaaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatactttgataaccgtgaagaaagaacaagatagaag